(19) United States
(12) Patent Application Publication
(10) Pub. No.: US 2024/0309328 A1
(43) Pub. Date: Sep. 19, 2024

(54) Use of Fibroblast Growth Factor-8 For Tissue Regeneration

(71) Applicant: UNIVERSITY OF CONNECTICUT, Farmington, CT (US)

(72) Inventors: Cato T. Laurencin, Farmington, CT (US); Paulos Mengsteab, Farmington, CT (US); Takayoshi Otsuka, Farmington, CT (US)

(21) Appl. No.: 18/270,368

(22) PCT Filed: Jan. 4, 2022
(86) PCT No.: PCT/US2022/011109

§ 371 Date: Jun. 29, 2023

Related U.S. Application Data
(60) Provisional application No. 63/134,062, filed on Jan. 5, 2021.

(51) Int. Cl. C12N 5/077; A61K 38/00; C07K 14/50

(52) U.S. Cl. CPC C12N 5/0658; C07K 14/50; C12N 5/0655; A61K 38/00; C12N 2501/119; C12N 2506/1323; C12N 2506/1384

(57) ABSTRACT

Methods for enhancing myogenic and/or chondrogenic lineage commitment of stem cells, inhibiting adipogenic differentiation of stem cells, and for promoting tissue regeneration using fibroblast growth factor 8 (FGF8).

### CROSS REFERENCES

This application claim priority to U.S. Provisional Patent Application Ser. No. 63/134,062 filed Jan. 5, 2021, incorporated by reference here in its entirety.

### STATEMENT OF GOVERNMENT SUPPORT

This invention was made with government support under Grant No. DPIAR068147 awarded by the National Institutes of Health. The government has certain rights in the disclosure.

### SEQUENCE LISTING STATEMENT

A computer readable form of the Sequence Listing is filed with this application by electronic submission and is incorporated into this application by reference in its entirety. The Sequence Listing is contained in the file created on Jan. 3, 2022 having the file name “21-0001-WO-SeqList_ST25.txt” and is 15 kb in size.

### BACKGROUND

Regeneration of complex tissues and organs, such as the limb, is well documented in lower vertebrates (e.g. fish and salamanders), which exhibit robust regeneration potential to regrow appendages after amputation (Brockes and Kumar, 2005: Tanaka, 2016). Mammals, in contrast, have a limited capacity for complex tissues and organ regeneration (Han et al., 2005; Laurencin and Nair, 2015). Therefore, understanding the limitation of regeneration in mammals and improving it remains a significant goal for therapeutic development.

One of the unique and essential events during limb regeneration is blastema formation (McCusker et al., 2015; Stocum, 2017). The blastema is an undifferentiated heterogeneous cell population of lineage-restricted progenitor cells derived from connective tissue fibroblasts (Kragl et al., 2009; McCusker et al., 2016). Blastema formation requires an innervated wound epithelium, which functions to recruit blastema cells to the wound site and allows them to proliferate (McCusker et al., 2015; Stocum, 2017). In the absence of nerve signals, denervation of the appendage, regeneration fails to initiate or blastema size is reduced depending on the timing of denervation (Singer, 1978). Thus, it appears that nerve signaling is required to maintain cells in an undifferentiated and proliferative state until the final pattern and size of the missing limb is restored (Farkas and Monaghan, 2017: Satoh et al., 2018).

Although blastema formation is a key event during complex tissue regeneration, induction and maintenance of blastema formation in mammals remains challenging.

### SUMMARY DISCLOSURE

In a first aspect, the disclosure provides a method for enhancing myogenic and/or chondrogenic lineage commitment of mammalian mesenchymal stem cells (MSCs), including, but not limited to, adipose derived stem cells (ADSCs), and/or muscle progenitor cells (MPCs), comprising contacting the MSCs, ADSCs, and/or MPCs with an amount effective of fibroblast growth factor 8 (FGF8) to enhance myogenic and/or chondrogenic lineage commitment of the MSCs, ADSCs, and/or MPCs.

In a second aspect, the disclosure provides a method for suppressing adipogenic and/or tenogenic differentiation of MSCs, including, but not limited to, ADSCs, and/or MPCs, comprising contacting mammalian MSCs, ADSCs, and/or MPCs with an amount effective of fibroblast growth factor 8 (FGF8) to suppress adipogenic and/or tenogenic differentiation of the MSCs, ADSCs, and/or MPCs.

In a third aspect the disclosure provides a method for promoting tissue regeneration in a mammalian subject, such as a human subject, comprising administering an amount effective of fibroblast growth factor 8 (FGF8) to regenerate tissue in the mammalian subject.

In a fourth aspect the disclosure provides a method for promoting myofiber formation in a mammalian subject, such as a human subject, comprising administering an amount effective of fibroblast growth factor 8 (FGF8) to promote myofiber formation in the mammalian subject.

In a fifth aspect the disclosure provides a method for promoting myogenesis in a mammalian subject, such as a human subject, comprising administering an amount effective of fibroblast growth factor 8 (FGF8) to promote myogenesis in the mammalian subject.

In a sixth aspect the disclosure provides a method for treating a musculoskeletal disorder, comprising administering an amount effective of fibroblast growth factor 8 (FGF8) to a mammalian subject, such as a human subject, having a musculoskeletal disorder, to treat the disorder.

### BRIEF DESCRIPTION OF FIGURES

FIG. 1 shows FGF8-b supplementation enhances cell proliferation in rat ADSCs and MPCs. (A) MTS assay and (B) DNA quantification of rat ADSCs cultured in growth medium (GM) supplemented with or without FGF-8b at Day 1, 3, 7, and 14. (C) DNA quantification of rat ADSCs cultured in growth and differentiation medium. (D) DNA quantification of rat MPCs in myogenic medium at Day 14. Statistical analyses: one-way ANOVA (A, B), unpaired Student's t-test (C, D). * P<. 05. ** P<01. *** P<001, and P<. 0001.

FIG. 2 shows FGF-8b supplementation alters FGF downstream signaling gene expression and differentiation potentials. (A) mRNA expression of Fgf2 and FGF receptors. (B) mRNA expression of FGF signaling downstream genes. (C) mRNA expression of lineage specific transcription factors. All data are expressed as fold change relative to both the housekeeping gene and growth medium control (without FGF-8b supplementation) condition. Statistical analyses: unpaired Student's t-test (A, B, C). * P<05, ** P<01, *** P<001, and **** P<. 0001.

FIG. 3 shows FGF-8b does not affect osteogenesis in rat ADSCs. (A, B) Representative images of the stained calcium deposit after 21 days of culture. Treatment conditions were (A) osteogenic medium and (B) osteogenic medium supplemented with 100 ng/mL FGF-8b. Scale bar=100 μm. (C) Quantification of the stained calcium deposit were performed using the eluted Alizarin S stain via measuring absorbance at 450 nm. The readings were normalized to background values of non-induced control ADSCs. (D) mRNA expression of osteogenic markers. Data are expressed as fold change relative to both the housekeeping gene and osteogenic medium condition.

FIG. 4 shows FGF-8b enhances chondrogenesis in rat ADSCs. (A) Macroscopic aspect of micromasses after 21 days of culture in chondrogenic medium with and without FGF-8b supplementation. (B-D) Histological staining of micromass section after 21 days of culture: (B, C) hematoxylin/eosin staining: (D, E) alcian/nuclear fast staining. Scale bar=100 μm. (F) mRNA expression of chondrogenic markers. Data are expressed as fold change relative to both the housekeeping gene and chondrogenic medium condition. Statistical analyses: unpaired Student's t-test (F). ** P<01.

FIG. 5 shows FGF-8b suppresses adipogenesis in rat ADSCs. (A, B) Representative images of the stained lipid droplets after 14 days of culture. Treatment conditions were (A) adipogenic medium and (B) adipogenic medium supplemented with 100 ng/ml FGF-8b. Scale bar=100 μm. (C) Quantification of the stained lipid droplets were performed using the eluted Oil O stain via measuring absorbance at 510 nm. The readings were normalized to background values of non-induced control ADSCs. (D) mRNA expression of adipogenic markers. Data are expressed as fold change relative to both the housekeeping gene and adipogenic control condition. Statistical analyses: unpaired Student's t-test (C, D). ** P<01, *** P<. 005, and **** P<. 0001.

FIG. 6 shows FGF-8b suppresses tenogenesis in rat ADSCs. (A-F) Representative fluorescent images stained with Scx, Tnmd, or TenC after 14 days of culture. Treatment conditions were (A, C, E) tenogenic medium and (B, D, F) tenogenic medium supplemented with 100 ng/ml FGF-8b. Scale bar: 100 μm. (G) mRNA expression of tenogenic markers. Data are expressed as fold change relative to both the housekeeping gene and tenogenic medium condition. Statistical analyses: unpaired Student's t-test (G). * P<. 05, ** P<01, and *** P<001.

FIG. 7 shows FGF-8b enhances myogenesis in rat MPCs. (A, B) Representative fluorescent images stained with Desmin after 14 days of culture. Treatment conditions were (A) myogenic medium and (B) myogenic medium supplemented with 100 ng/ml FGF-8b. Scale bar=2 mm. (C) mRNA expression of myogenic markers. Data are expressed as fold change relative to both the housekeeping gene and myogenic control condition. Statistical analyses: unpaired Student's t-test (C). * P<. 05 and ** P<01.

FIG. 8 shows (A) Schematic images of cell separation method. (B) Morphology of FIBs (C) Morphology of MPCs. (D) MTS assay showed different metabolic activities between FIBs and MPCs. (E, F, G) FIBs are positive for Vimentin and αSMA (fibroblast marker) and PDGFRα (FAPs marker). (H, I, and J) MPCs are positive for Pax7 (SCs marker), MyoD (myogenic progenitor marker), and negative for desmin (differentiated myoblast marker). (K) PDGFRα positive cells are enriched in FIBs, and Pax7 and MyoD positive cells are enriched in MPCs. Scale bar: 100 μm.

FIG. 9 shows (A) Tenascin C (tenocyte marker) expression decreased at FGF8b dose dependent manner. (B) DNA amount tended to increase at FGF8b dose dependent manner. (C) Tenogenic marker genes (Scx, Tnmd, TenC, Col1al, Col3al) decreased at FGF8b dose dependent manner. (D) FGF8b treatment altered the ratio of col1 and Col3 that showed anti-fibrosis response. Scale bar: 100 μm.

FIG. 10 shows (A) Oil O staining showed the suppression of adipocyte differentiation at FGF8b dose dependent manner. (B) MHC staining showed enhanced myofiber formation in FIBs at FGF8b dose dependent manner. (C) DNA amount tended to increase at FGF8b dose dependent manner. (D) Quantification of Oil O staining. (E) Adipogenic marker genes (Pparg, fabp4, adipoq and leptin) decreased at FGF8b dose dependent manner. FAPs marker gene (PDGFRα) did not alter. Scale bar: 100 μm.

FIG. 11 shows (A) MHC staining showed enhanced myofiber formation in MPCs at FGF8b dose dependent manner. (B) DNA amount tended to increase at FGF8b dose dependent manner. (C) Quantification of MHC positive area. (D) Both satellite cell marker (Pax7) and myogenic marker genes (MyoD, MyoG, Desmin, and Tnnt1) increased at FGF8b dose dependent manner. Scale bar: 100 μm (high mag) and 2 mm (low mag).

FIG. 12 shows representative images of PDGFRα, Pax7, and MyoD staining in FIBs and MPCs.

FIG. 13 shows MPCs did not differentiate into tenocytes and adipocytes (top). Enhanced myofiber formation with FGF8b treatment were observed both tenogenic and adipogenic conditions (bottom).

FIG. 14 shows a graphical abstract of the disclosure.

FIG. 15 shows representative images of (A) Pax7 and (B) MyoD staining in Fibroblasts (FIBs) and MPCs.

FIG. 16 shows (A) Oil O staining showed the suppression of adipocyte differentiation at a FGF8b dose dependent manner and MHC staining showed enhanced myofiber formation in FIBs at FGF8b dose dependent manner. (B) Oil O staining showed MPCs did not differentiate into adipocytes and MHC staining showed enhanced myofiber formation in MPCs at a FGF8b dose dependent manner.

FIG. 17 shows the characterization of rat ADSCs. (A) The morphology of rat ADSCs at passage 3. (B-F) ADSCs were analyzed for cell surface markers at passage 3. (G-I) ADSCs at passage 3 were differentiated into osteocyte, adipocyte, and chondrocyte. Calcium deposits were stained with Alizarin S (G), Lipid droplets were visualized by oil O staining (H), and proteoglycans were stained by Alcian staining (I). Inserts represent ADSCs, cultured in Growth medium as negative controls. Scale bar: 100 μm.

FIG. 18 shows the characterization of rat MPCs. (A-C) Isolated MPCs were positive for Pax7 (A), MyoD (B), and Desmin (C). (D-F) MPCs were analyzed for cell surface markers at passage 2. Scale bar: 100 μm.

FIG. 19 shows the characterization of rat ADSCs after induction of myogenic differentiation. (A-D) Representative fluorescent images stained with MyoD or Desmin after 14 days of culture. Treatment conditions were (A, C) control and (B, D) 100 ng/ml FGF-8b. Scale bar=100 μm.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala: A), asparagine (Asn: N), aspartic acid (Asp: D), arginine (Arg: R), cysteine (Cys: C), glutamic acid (Glu: E), glutamine (Gln: Q), glycine (Gly: G), histidine (His: H), isoleucine (Ile: I), leucine (Leu: L), lysine (Lys: K), methionine (Met: M), phenylalanine (Phe: F), proline (Pro: P), serine (Ser: S), threonine (Thr: T), tryptophan (Trp: W), tyrosine (Tyr: Y), and valine (Val: V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense: that is to say, in the sense of “including, but not limited to”. Additionally, the words “herein,” “above,” and “below” and words of similar import, when used in this application, shall refer to this application as a whole and not to any particular portions of the application.

In a first aspect, the disclosure provides a method for enhancing myogenic and/or chondrogenic lineage commitment of mammalian mesenchymal stem cells (MSCs), including, but not limited to, adipose derived stem cells (ADSCs), and/or muscle progenitor cells (MPCs), comprising contacting the MSCs, ADSCs, and/or MPCs with an amount effective of fibroblast growth factor 8 (FGF8) to enhance myogenic and/or chondrogenic lineage commitment of the MSCs, ADSCs, and/or MPCs.

As used herein, “myogenic lineage commitment” is a developmental process by which mesenchymal stem cells, including, but not limited to ADSCs, and MPCs differentiate into muscle cells (myocytes) and “chondrogenic lineage commitment” is a developmental process by which mesenchymal stem cells, including, but not limited to ADSCs, differentiate into cartilage cells (chondrocytes). In one non-limiting embodiment the mesenchymal stem cells and/or MPCs are mammalian cells, including, but not-limited to, human cells.

As used herein, “mesenchymal stem cells (MSCs)” are multipotent stem cells that can differentiate into a variety of cell types, including bone cells (osteoblasts), cartilage cells (chondrocytes), tendon cells (tenocytes), neuronal cells, muscle cells (myocytes) and fat cells that give rise to marrow adipose tissue (adipocytes).

As used herein, “adipose derived stem cells (ADSCs)” are a type of mesenchymal stem cells (MSCs) that are obtained from adipose tissue.

As used herein, “muscle progenitor cells (MPCs)” are satellite stem cells-rich cell population which can differentiate into skeletal muscle cells.

In a second aspect, the disclosure provides a method for suppressing adipogenic and/or tenogenic differentiation of MSCs, including, but not limited to, ADSCs, and/or MPCs, comprising contacting mammalian MSCs, ADSCs, and/or MPCs with an amount effective of fibroblast growth factor 8 (FGF8) to suppress adipogenic and/or tenogenic differentiation of the MSCs, ADSCs, and/or MPCs.

As used herein “adipogenic differentiation” is a developmental process by which mesenchymal stem cells differentiate into adipose tissue (adipocytes) and “tenogenic differentiation” is a developmental process by which stem cells differentiate into tendon-like cells.

As used herein, “fibroblast growth factor 8 (FGF8)” is any isoform of the FGF8 family. In one non-limiting embodiment, the FGF8 comprises a mammalian FGF8. In other embodiment, the FGF8 comprises a human FGF8, including FGF8a, FGF8c, FGF8b, FGF8f, and functional fragments thereof. In specific embodiments, the FGF8 comprises the amino acid sequence of any one of SEQ ID NOS: 1-7 (wherein residues in parentheses such as the N-terminal methionine, s are optional and may be present or absent), or functional fragments thereof. In one embodiment, the FGF comprises FGF8b, or a functional fragment thereof. In another embodiment, the FGF8 comprises the amino acid sequence of SEQ ID NO:3, wherein the N-terminal methionine may be present or absent: or functional fragments thereof. In another, non-limiting embodiment, the FGF8 comprises an amino acid fragment of residues 23 to 215 of SEQ ID NOs: 3, 5 and/or 7. In other embodiments, the FGF8 comprises an amino acid fragment of residues 23 to 209 of SEQ ID NOs: 1 or 4, residues 23 to 204 of SEQ ID NO: 2, and/or residues 23 to 190 of SEQ ID NO: 6. In various examples of this embodiment, the FGF8 protein or protein fragment comprises an optional N-terminal methionine.

In a third aspect the disclosure provides a method for promoting tissue regeneration in a mammalian subject, such as a human subject, comprising administering an amount effective of fibroblast growth factor 8 (FGF8) to regenerate tissue in the mammalian subject.

In a fourth aspect the disclosure provides a method for promoting myofiber formation in a mammalian subject, such as a human subject, comprising administering an amount effective of fibroblast growth factor 8 (FGF8) to promote myofiber formation in the mammalian subject.

In a fifth aspect the disclosure provides a method for promoting myogenesis in a mammalian subject, such as a human subject, comprising administering an amount effective of fibroblast growth factor 8 (FGF8) to promote myogenesis in the mammalian subject.

In a sixth aspect the disclosure provides a method for treating a musculoskeletal disorder, comprising administering an amount effective of fibroblast growth factor 8 (FGF8) to a mammalian subject, such as a human subject, having a musculoskeletal disorder, to treat the disorder.

Human FGF8e
(SEQ ID NO: 1)
(M) GSPRSALSCLLLHLLVLCLQAQEGPGRGPALGRELASLFRA
GREPQGVSQQHVREQSLVTDQLSRRLIRTYQLYSRTSGKHVQVLA
NKRINAMAEDGDPFAKLIVETDTEGSRVRVRGAETGLYICMNKKG
KLIAKSNGKGKDCVFTEIVLENNYTALQNAKYEGWYMAFTRKGRP
RKGSKTRQHQREVHEMKRLPRGHHTTEQSLRFEFLNYPPFTRSLR
GSQRTWAPEPR
Human FGF8a
(SEQ ID NO: 2)
(M) GSPRSALSCLLLHLLVLCLQAQHVREQSLVTDQLSRRLIRT
YQLYSRTSGKHVQVLANKRINAMAEDGDPFAKLIVETDTFGSRVR
VRGAETGLYICMNKKGKLIAKSNGKGKDCVFTEIVLENNYTALQN
AKYEGWYMAFTRKGRPRKGSKTRQHQREVHEMKRLPRGHHTTEQS
LRFEFLNYPPFTRSLRGSQRTWAPEPR
Human FGF8b
(SEQ ID NO: 3)
(M) GSPRSALSCLLLHLLVLCLQAQVTVQSSPNETQHVREQSLV
TDQLSRRLIRTYQLYSRTSGKHVQVLANKRINAMAEDGDPFAKLI
VETDTFGSRVRVRGAETGLYICMNKKGKLIAKSNGKGKDCVFTEI
VLENNYTALQNAKYEGWYMAFTRKGRPRKGSKTRQHQREVHEMKR
LPRGHHTTEQSLRFEFLNYPPFTRSLRGSQRTWAPEPR
Human FGF8f
(SEQ ID NO: 4)
(M) GSPRSALSCLLLHLLVLCLQAQEGPGRGPALGRELASLFRA
GREPQGVSQQVTVQSSPNFTQHVREQSLVTDQLSRRLIRTYQLYS
RTSGKHVQVLANKRINAMAEDGDPFAKLIVETDTFGSRVRVRGAE
TGLYICMNKKGKLIAKSNGKGKDCVFTEIVLENNYTALQNAKYEG
WYMAFTRKGRPRKGSKTRQHQREVHEMKRLPRGHHTTEQSLRFEF
LNYPPFTRSLRGSQRTWAPEPR
Mouse FGF8
(SEQ ID NO: 5)
(M) GSPRSALSCLLLHLLVLCLQAQVTVQSSPNFTQHVREQSLV
TDQLSRRLIRTYQLYSRTSGKHVQVLANKRINAMAEDGDPFAKLI
VETDTFGSRVRVRGAETGLYICMNKKGKLIAKSNGKGKDCVFTEI
VLENNYTALQNAKYEGWYMAFTRKGRPRKGSKTRQHQREVHFMKR
LPRGHHTTEQSLRFEFLNYPPFTRSLRGSQRTWAPEPR
Rabbit FGF8b
(SEQ ID NO: 6)
(M) GSPRSALSCLLLHLLVLCLQAQVTVQSSPNETQHVREQSLV
TDQLSRRLIRTYQLYSRTSGKHVQVLANKRINAMAEDGDPFAKLI
VETDTFGSRVRVRGAETGLYICMNKKGKLIAKSNGKGKDCVFTEI
VLENNYTALQNAKYEGWYMAFTRKGRPRKGSKTRQHQREVHFMKR
LPRGHHTTEQSLRFEFLNYP
Horse FGF8
(SEQ ID NO: 7)
(M) GSPRSALSCLLLHLLVLCLQAQVRRTARRRGPGGRRGTPVD
NVGQGHEDGPCGRRGRAGRNLSSPAPNYPEEGAKEQRGSALPKVT
QQHVREQSLVTDQLSRRLIRTYQLYSRTSGKHVQVLANKRINAMA
EDGDPFAKLIVETDTEGSRVRVRGAETGLYICMNKKGKLIAKSNG
KGKDCVFTEIVLENNYTALQNAKYEGWYMAFTRKGRPRKGSKTRQ
HQREVHFMKRLPRGHHTTEQSLRFEFLNYPPFTRSLRGSQRTWAP
EPR

The contacting of the FGF8 to the MSCs, ADSCs, and/or MPCs can occur using any method suitable for use with the disclosure, including, but not limited to contacting which occurs in vitro or in vivo. In one embodiment, the contacting is carried out in vitro, and the methods can be used, for example, to induce robust proliferation of MSC, ADSCs, and/or MPCs in vitro. Such in vitro methods may further be used to enhance in vitro chondrogenic differentiation and suppress adipogenic and tenogenic differentiation in ADSCs; or to enhance myofiber formation in MPCs in vitro. In other embodiments, the contacting occurs in vivo in a mammalian subject. In this embodiment, the methods may comprising administering FGF8 to any suitable mammalian subject, including but not limited to a horse, rabbit, mouse, cow, dog, cat, sheep, goat, or human. In one embodiment, the mammalian subject may be a human. In another, non-limiting embodiment, the mammalian subject can be a horse.

As used herein an “effective amount” for in vivo use can by any amount or concentration, which is effective to carry out the methods of the disclosure. Any suitable dosage range may be used as determined by attending medical personnel. Dosage regimens can be adjusted to provide the optimum desired response (e.g., a therapeutic or prophylactic response). A suitable dosage range may, for instance, be 0.1 ug/kg-100 mg/kg body weight: alternatively, it may be 0.5 ug/kg to 50 mg/kg: 1 ug/kg to 25 mg/kg, or 5 ug/kg to 10 mg/kg body weight. In some embodiments, the recommended dose could be lower than 0.1 mcg/kg, especially if administered locally. In other embodiments, the recommended dose could be based on weight/m² (i.e. body surface area), and/or it could be administered at a fixed dose (e.g., 0.05-100 mg). The FGF8 can be delivered in a single bolus, or may be administered more than once (e.g., 2, 3, 4, 5, or more times) as determined by an attending medical personnel.

The FGF8 may be the sole active agent administered to the subject, or may be administered together (in a single formulation or separately) with one or more other active agents suitable for an intended use. In various non-limiting embodiments, the FGF8 is added to any type of culture medium suitable for use according to the methods of the disclosure. Types of culture medium include, but are not limited to, growth medium, osteogenic medium, adipogenic medium, and/or chondrogenic medium. In other non-limiting embodiments, growth medium can be supplemented with any additional active agents suitable for use in the medium, including, but not limited to, dexamethasone, ascorbic acid, beta-glycerophosphate, IBMX, rh-insulin, indomethacin, ascorbate-2-phosphate, sodium pyruvate, ITS, and/or TGF-β3.

As used herein “enhance” is defined as any increased myogenic and/or chondrogenic lineage commitment of the MSCs, ADSCs, and/or MPCs. In various non-limiting embodiments, an increase in the total number of MSCs, ADSCs, and/or MPCs committed to the myogenic and/or chondrogenic lineage is assessed using marker gene expression, histological and/or immunohistochemical measurement.

As used herein “suppressing” is defined as any decrease of the adipogenic and/or tenogenic differentiation of the MSCs and/or ADSCs.

The methods of the disclosure involving in vivo administration can, for example, promote tissue regeneration. As used herein “tissue regeneration” means any renewal, growth, and/or repair of tissue that is damaged, or suffers from a disease or disorder. In various embodiments, the mammalian subject has a disease, or disorder, which results in tissue damage, including but not limited to a musculoskeletal disorders or injury. Examples of musculoskeletal disorders or injuries that can be treated using the methods of the disclosure include, but are not limited to, lacerations, contusions, strains, inflammation, or degenerative diseases of the muscles, tendons, or skeletal system. In one non-limiting embodiment, the subject has osteoarthritis, which may include inflammation of the knee joint or paraspinal muscles. In various other embodiments, the subject has an injury including but not limited to rotator cuff, elbow; ankle, spine, and/or wrist injuries.

In one embodiment, the subject has osteoarthritis and method results in reduction of adipose induced inflammation in the knee joint, spinal muscles, or paraspinal muscles. This embodiment can comprise administering the FGF8 to or proximal to the knee joint or spinal muscles, or to fat pads of the knee joint or spinal muscles. Adipokines such as adiponectin and leptin are inflammatory cytokines released from adipose tissues. FGF8 treatment to suppress adipogenesis reduces adipokines release.

As used herein, the terms “treatment”, “treating”, and the like, refer to obtaining a desired pharmacologic and/or physiologic effect. The effect may be therapeutic in terms of a partial or complete cure for a disease and/or adverse symptoms attributable to the disease. “Treatment”, as used herein, covers any treatment of the recited disorders, particularly in a human, and includes: (a) limiting development of symptoms or flares from occurring in a subject having the disorder: (b) limiting worsening of symptoms or flares in a subject having the disorder: (c) inhibiting the disorder, i.e., arresting development of the disorder: (d) relieving the disorder, i.e., causing regression of the disorder. Treatment can be assessed using any suitable methods including, but not limited to, imaging, fluid sampling, assessment of inflammatory markers, and/or improvement in physical activity.

The FGF8 can be administered via any route suitable for an intended use. In certain embodiments, the FGF8 may be administered, e.g. orally, through injection by intravenous, intraperitoneal, intracerebral (intra-parenchymal), intracerebroventricular, intramuscular, subcutaneously, intraportal, or intralesional routes: by sustained release systems or by implantation devices. In certain embodiments, the FGF8 can be administered by bolus injection or continuously by infusion, or by implantation device directly into the injury or in the area surrounding (distal or proximal) the injury. In various embodiments, the FGF8 can be administered continuously via infusion for 7 to 14 days or once daily as a single bolus each day for 14 days. In one non-limiting embodiment, the FGF8 can be administered as a single bolus at the conclusion of surgery.

In all aspects and embodiments, particularly in vivo embodiments, the FGF8 may be administered as part of a pharmaceutical composition, comprising the FGF8 and a pharmaceutically acceptable carrier. The pharmaceutical composition may comprise in addition to FGF8, (a) a lyoprotectant: (b) a surfactant: (c) a bulking agent: (d) a tonicity adjusting agent: (e) a stabilizer: (f) a preservative and/or (g) a buffer. In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride. In other non-limiting embodiments, the composition can include heparan sulfate and/or other FGF proteins, including, but not limited, to FGF2.

### EXAMPLES

### Material and Methods

Reagents. Recombinant growth factors were obtained from R&D Systems (Minneapolis, Minnesota): human FGF-2 (233-FB), FGF-8b (423-F8), transforming growth factor-β3 (TGF-β3, 243-B3), platelet-derived growth factor-BB (PDGF-BB, 220-BB), and mouse growth differentiation factor 6 (GDF-6, 855-G6). Dexamethasone (D4902), L-ascorbic acid (A4544), beta-glycerophosphate (G9422), 3-isobutyl-1-methylxanthine (IBMX, 17018), recombinant human Insulin (rh-insulin, 12643), indomethacin (18280), and ITS (13146) were obtained from Millipore Sigma (St. Louis, Missouri). Fetal bovine serum (FBS, 16000044), horse serum (16050122), penicillin-streptomycin (pen/strep, 15140122), and sodium pyruvate (11360070) were obtained from Gibco™, Thermo Fisher Scientific (Waltham, Massachusetts).

Adipose derived stem cells (ADSCs) and muscle progenitor cells (MPCs) isolation and culture. ADSCs and MPCs were isolated from Sprague-Dawley rats (age: 16-18 weeks, weight: 450-550 grams). Rats were euthanized with CO₂ inhalation followed by neck dislocation. Inguinal fat pads were harvested and minced into small pieces. An equal volume of collagenase type I (0.2%, w/v) (Gibco™) in Hank's balanced salt solution was added to the fat tissue and agitated at 37° C. for 90 minutes. The cell suspension was filtered through a 70 μm filter for the removal of solid aggregates. The cells were plated in 10 cm dishes containing DMEM-F12 with 10% FBS and 1% pen/strep (growth medium: GM), then placed in a 37° C.-5% CO₂ incubator. The media was changed after 72 hours. The cells were passaged at 80-90% confluence. The GM was changed every two to three days and cells were expanded to passage 3 for this study.

For MPCs isolation, gastrocnemius muscles were harvested and minced into small pieces. The muscle tissues were digested in a mix of collagenase type I (0.2%, w/v) and dispase II (0.4%, w/v) (Sigma) in DMEM-F12 at 37° C. for 120 min. The cell suspension was filtered through a 100 μm filter for the removal of solid aggregates. The cells were plated in 10 cm dishes containing DMEM-F12 with 20% FBS, 25 ng/ml of FGF-2 and 1% pen/strep. To remove faster growing non-muscle cells, the supernatant containing non-adhered MPCs were transferred to Matrigel (Corning, Glendale, Arizona) coated 10 cm dishes after 24 hours. The cells were passaged after 80% confluence. The culture media was changed every three to four days and the cells were expanded to passage 2 for this study.

Flow cytometry. Rat ADSCs and MPCs were characterized by flow cytometry (MACSQuant® Analyzer 10: Miltenyi Biotec, Bergisch Gladbach, Germany). Rat ADSCs, passage three, and MPCs, passage two, (5×10⁵ cells per sample) were collected and washed thrice in sterile PBS containing 1% FBS, then incubated on ice with fluorescein isothiocyanate (FITC)-conjugated anti-rat CD90, CD29 (integrin beta 1), CD45, CD11b (BD BioSciences, San Jose, California), and CD31 (abcam, Cambridge, Massachusetts) at a concentration of 10 ug/mL for 30 min in the dark at 4° C. Unlabeled cells were used as controls. Flow Jo™ software (Treestar, Inc., Ashland, Oregon) was used to create the histograms.

MTS assay. The metabolic activity and proliferation of cells were assessed using the CellTiter® 96 AQueous nonradioactive cell proliferation assay (MTS assay: Promega, Madison, Wisconsin) following the manufacturer's protocol. Briefly, cells were washed with PBS, then MTS reagent in a ratio of 5:1 (media: MTS) added to each well. The plates were incubated for 1 hour at 37° C. The absorbance of the resulting solution was read at 490 nm using a microplate reader.

DNA Quantification. DNA was isolated and quantified using the Quant-iT™ PicoGreen dsDNA assay kit (Invitrogen, Carlsbad, California) following the manufacturer's instructions. Briefly, cell lysates were collected and mixed with the Quant-iT™ PicoGreen reagent, measured via spectrophotometry at 535 nm with excitation at 485 nm, and DNA content was quantified using a standard curve.

Multi-lineage differentiation induction. Rat ADSCs at passage 3 or MPCs at passage 2 were used to verify the differentiation capacity. Cells were seeded in 6 well plates at 1.5×10⁵ cells/well or 24 well plates at 3.0×10⁴ cells/well and grown to at least 80% confluence before being cultured in the differentiation medium.

To induce osteogenic differentiation, rat ADSC were cultured up to 21 days in Growth medium (GM) supplemented with 100 nM dexamethasone, 50 g/mL L-ascorbic acid, and 10 mM beta-glycerophosphate (Osteogenic medium). Mineralization of the extracellular matrix was visualized by staining with Alizarin S on day 21. The cells were fixed in 10% formalin for 10 minutes, and then incubated for 20 minutes in 1% Alizarin S (pH 4.2). After qualitative analysis by microscopy; the stain was eluted by 10% cetylpyridinium chloride for 15 minutes and analyzed at 450 nm absorbance.

To induce adipogenic differentiation, rat ADSC were cultured for 14 days in GM supplemented with 0.5 mM IBMX, 1 μM dexamethasone, 10 μg/mL rh-insulin, and 100 μM indomethacin (Adipogenic medium). Adipogenic differentiation was confirmed by staining with Oil-Red O at 14 day. The cultures were fixed in 10% formalin for 10 minutes, and then incubated for 20 minutes in Oil-Red O solution. After qualitative analysis by microscopy; the stain was eluted by 100% isopropanol for 10 minutes and analyzed at 510 nm absorbance.

To induce chondrogenic differentiation, rat ADSCs were cultured in GM supplemented with 1% ITS, 100 nM dexamethasone, 50 μg/mL ascorbate-2-phosphate, 0.9 mM sodium pyruvate, and 10 ng/ml TGF-β3 (Chondrogenic medium). For micromass culture, a 20 μL drop, containing 5.0×10⁵ cells, was carefully placed in the center of each well of a 24-well plate. The cell drops were incubated for 2 hours at 37° C. and 5% CO₂, subsequently CM was carefully added to each well. The micromass pellets were then cultured for 21 days in CM. The pellets were fixed in 10% formalin for 24 hours, embedded in paraffin, cut into 5 μm sections and stained with standard hematoxylin/cosin and 1% alcian (pH 1.0)/0.1% nuclear fast.

To induce tenogenic differentiation, rat ADSCs were cultured for 14 days in GM supplemented with 50 ng/ml GDF-6, and 10 ng/ml PDGF-BB (Tenogenic medium) (Norelli et al., 2018).

To induce myogenic differentiation, rat ADSCs or MPCs were cultured for 14 days in DMEM-F12 supplemented with 5% horse serum and 1% pen/strep (Myogenic medium).

Immunofluorescence. Cells were rinsed with PBS, fixed with 4% paraformaldehyde in PBS for 20 min at room temperature and permeabilized with 0.1% Triton™ X 100 for 10 min at room temperature. Then, cells were blocked by 1% bovine serum albumin (Sigma) and incubated with primary antibodies overnight at 4° C. Thereafter, cells were rinsed thrice with PBS and incubated with secondary antibody for 2 h at room temperature in the dark. Primary antibodies used were anti-SCXA (1:250, abcam), anti-tenomodulin (1:100, abcam), anti-Tenascin C (1:200, Novus Biologicals, Littleton, Colorado), anti-Pax7 (1:200, LSBio, Seattle, Washington), anti-MYOD (1:200, LSBio), and anti-Desmin (1:100, Invitrogen). DAPI (Invitrogen) was used as nuclear staining. As for secondary antibodies, Goat Anti-Rabbit IgG H&L (Alexa Fluor® 488) and Goat Anti-mouse IgG H&L (Alexa Fluor® 594) were used. All stained samples were examined under a Leica DMi8 inverted microscope (Leica Microsystems, Wetzlar, Germany).

Quantitative real-time PCR (qRT-PCR). Total RNA was isolated using the RNeasy™ Mini Kit (QIAGEN, Alameda, California) according to the manufacturer's instructions. 1-4 μg of total RNA was reverse-transcribed to cDNA by using RNA to cDNA EcoDry™ Premix (Clontech, Palo Alto, California) in a total volume of 20 μL. Taqman™ predesigned primers (Thermo Fisher Scientific) (Table 1) were used for qRT-PCR, and the signal was detected by an iQ5TM real-time PCR machine (Bio-Rad, Hercules, California). The threshold cycle values of target genes were standardized against GAPDH expression and normalized to the expression in the control culture. The fold change in expression was calculated using the ΔΔCt comparative threshold cycle method. All qRT-PCRs were run in triplicate.

TABLE 1
TaqMan Assay ID used for quantitative real-time PCR.

| Gene | TaqMan | Entrez | | |
|---|---|---|---|---|
| Symbol | Assay ID | Gene ID | GenBank ID | Gene Name |
| Fgf8 | Rn00590996_m1 | 29349 | NM_133286.1 | fibroblast growth factor 8 |
| Fgf2 | Rn00570809_m1 | 54250 | NM_019305.2 | fibroblast growth factor 2 |
| Fgfr1 | Rn00577234_m1 | 79114 | NM_024146.1 | fibroblast growth factor receptor 1 |
| Fgfr2 | Rn01269940_m1 | 25022 | NM_012712.1 | fibroblast growth factor receptor 2 |
| Fgfr3 | Rn00584799_m1 | 84489 | NM_053429.1 | fibroblast growth factor receptor 3 |
| Fgfr4 | Rn01441815_m1 | 25114 | NM_001109904.1 | fibroblast growth factor receptor 4 |
| Etv4 | Rn01464037_m1 | 360635 | NM_001108299.1 | ets variant 4 |
| Etv5 | Rn00465814_g1 | 303828 | NM_001107082.1 | ets variant 5 |
| Spry4 | Rn01760706_m1 | 291610 | NM_001106150.1 | sprouty homolog 4 |
| Twist1 | Rn00585470_s1 | 85489 | NM_053530.2 | twist family bHLH transcription |
| | | | | factor 1 |
| Twist2 | Rn00572482_m1 | 59327 | NM_021691.2 | twist family bHLH transcription |
| | | | | factor 2 |
| Dusp6 | Rn00518185_m1 | 116663 | NM_053883.2 | dual specificity phosphatase 6 |
| Runx2 | Rn01512298_m1 | 367218 | NM_001278483.1 | runt-related transcription factor 2 |
| Sp7 | Rn02769744_s1 | 300260 | NM_001037632.1 | Sp7 transcription factor |
| Alpl | Rn01516028_m1 | 25586 | NM_013059.1 | alkaline phosphatase |
| Col1a1 | Rn01463848_m1 | 29393 | NM_053304.1 | collagen, type I, alpha 1 |
| Sox9 | Rn01751070_m1 | 140586 | XM_003750950.3 | SRY (sex determining region Y)- |
| | | | | box 9 |
| Acan | Rn00573424_m1 | 58968 | NM_022190.1 | Aggrecan |
| Pparg | Rn00440945_m1 | 25664 | NM_013124.3 | peroxisome proliferator-activated |
| | | | | receptor gamma |
| Fabp4 | Rn00670361_m1 | 79451 | NM_053365.1 | fatty acid binding protein 4 |
| Adipoq | Rn00595250_m1 | 246253 | NM_144744.3 | adiponectin |
| Scx | Rn01504576_m1 | 680712 | NM_001130508.1 | Scleraxis |
| Tnmd | Rn00574164_m1 | 64104 | NM_022290.1 | Tenomodulin |
| Tnc | Rn01454948_m1 | 116640 | NM_053861.1 | tenascin C |
| Col3a1 | Rn01437681_m1 | 84032 | NM_032085.1 | collagen, type III, alpha 1 |
| Myod1 | Rn00598571_m1 | 337868 | NM_176079.1 | myogenic differentiation 1 |
| Myog | Rn00567418_m1 | 29148 | NM_017115.2 | myogenin |
| Myf5 | Rn01502778_m1 | 299766 | NM_001106783.1 | myogenic factor 5 |
| Tnnt1 | Rn00592835_m1 | 171409 | NM_134388.2 | troponin T type 1 (skeletal, slow) |
| Gapdh | Rn01775763_g1 | 24383 | NM_017008.4 | glyceraldehyde-3-phosphate |
| | | | | dehydrogenase |

Statistical Analysis. GraphPad Prism™ 7 (GraphPad Software; San Diego, California) was used for statistical analysis and graph design. Results were expressed as the mean values #standard deviation. Comparisons between two groups were performed with the unpaired Student's t-test. Comparisons of more than two groups were done by one-way ANOVA with Dunnett's post-hoc test. Differences were considered significant if the p-value was <0.05. Statistical significance was shown with * P<05, ** P<01, *** P<001, and **** P<. 0001.

### Example 1: Rat ADSCs and MPCs Characterization

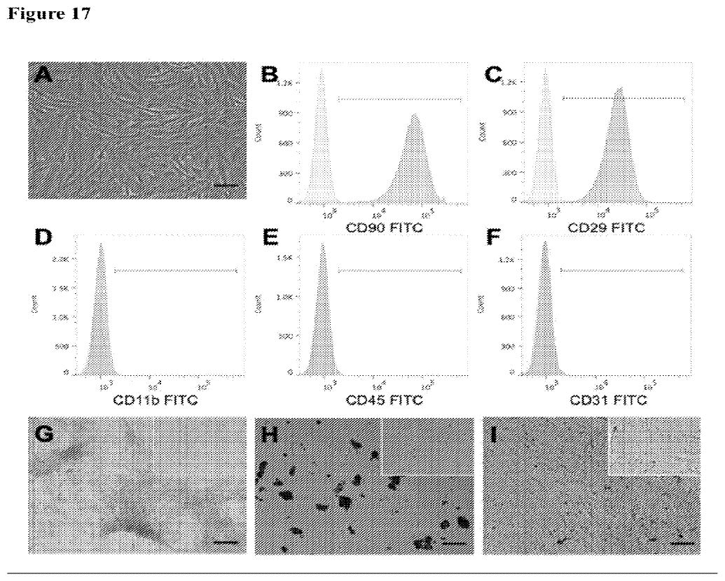

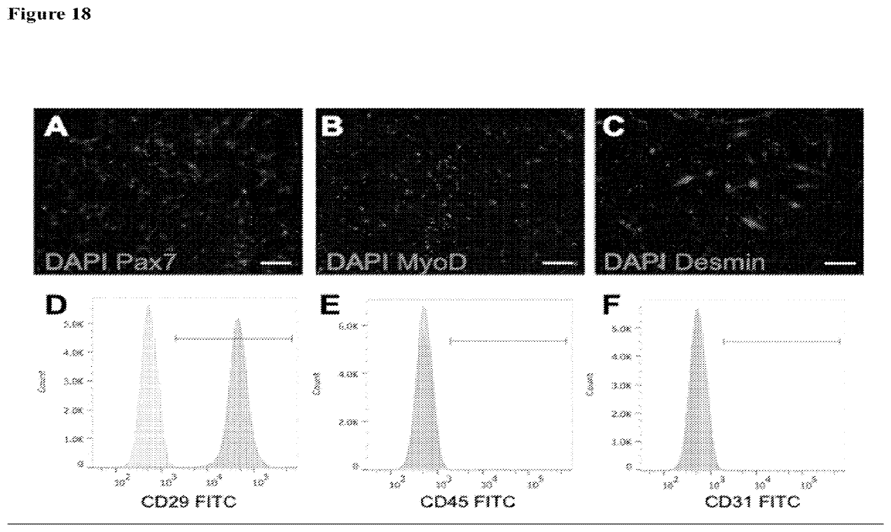

Isolated ADSCs showed a spindle-shaped and flat polygonal morphology (FIG. 17A). ADSCs at passage 3 were analyzed for expression of MSC specific cell-surface markers (FIG. 17B-F). Flow cytometry analysis showed that ADSCs were positive for the MSC surface markers CD90 (98.4%±2.8) and CD29 (99.2%±1.4), and negative for the hematopoietic stem cell surface markers CD11b (1.0%±0.2), CD45 (0.4%±0.3) and endothelial surface marker CD31 (0.8%±0.2). Rat ADSCs were successfully differentiated into multiple lineages such as bone, fat, and cartilage (FIG. 17 G-H). Primary rat MPCs at passage 2 were characterized by immunofluorescence and flow cytometry analysis. MPCs were positive for a satellite stem cell marker (Pax7), and myogenic markers (MyoD and Desmin) (FIG. 18A-C). Flow cytometry analysis showed that MPCs were positive for CD29 (99.9%±0.1), and negative for CD45 (0.1%±0.1) and CD31 (0.2%±0.1) (FIG. 18D-F).

### Example 2: FGF-8b Increases Proliferation in Cultured Rat ADSCs and MPCs

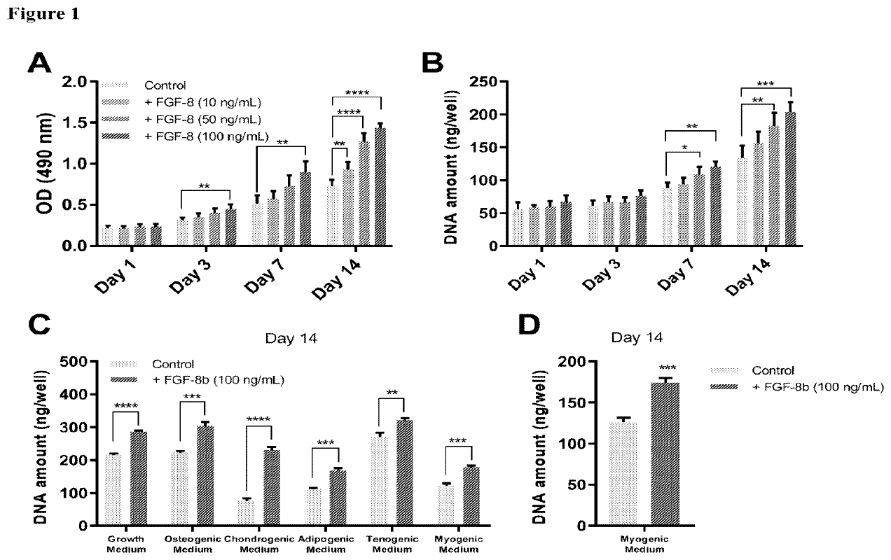

The ability of FGF-8b to promote proliferation of rat ADSCs was evaluated by metabolic and total DNA content analysis (FIG. 1). The metabolic activity of rat ADSCs was followed for 14 days, and metabolic activity was enhanced at day 3 of culture when GM was supplemented with 100 ng/ml of FGF-8b (FIG. 1A). At days 7 and 14, the metabolic activity of rat ADSCs supplemented with 100 ng/ml of FGF-8b continued to be upregulated in comparison to GM alone (FIG. 1A). The effects of lower concentrations of FGF-8b (10 ng/ml and 50 ng/mL) were only seen at day 14 (FIG. 1A). Total DNA content was surveyed as a surrogate for cell count, and an increase in total DNA content of rat ADSCs was observed at day 7 and 14 when the GM was supplemented with 50 or 100 ng/mL of FGF-8b (FIG. 1B). Furthermore, the mitogenic potential of rat ADSCs were evaluated in osteogenic, chondrogenic, adipogenic, tenogenic, and myogenic mediums. In all medium conditions, the supplementation of FGF-8b at 100 ng/mL was found to significantly increase DNA content (FIG. 1C). Finally, the total DNA was quantified in MPCs, and it was found that total DNA was significantly increased when MPCs were cultured in myogenic medium supplemented with 100 ng/ml of FGF-8b (FIG. 1D).

### Example 3: FGF-8b Regulates Expression of FGF Signaling Pathway Components in ADSCs

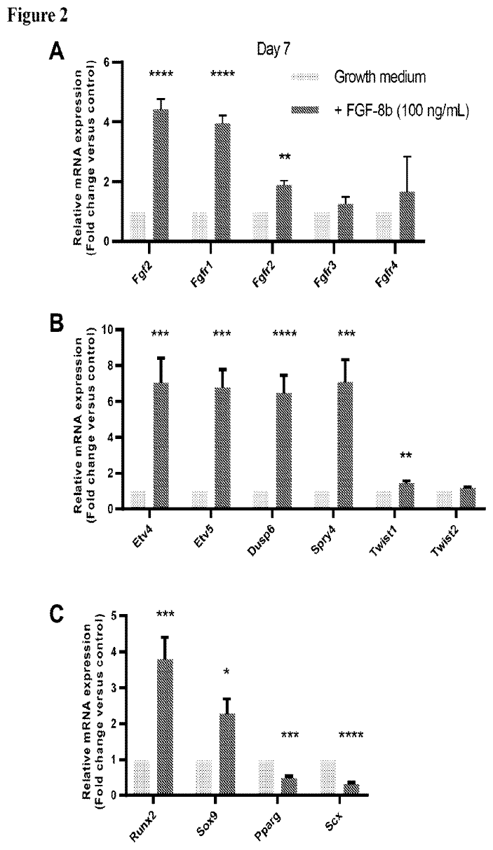

Following 7 days of culture, targets of the FGF-8 signaling pathway from the receptor to downstream signaling targets was evaluated. Endogenous mRNA expression of FGF-8 was not detected by qRT-PCR in growth medium with or without FGF-8b supplementation. FGF-8b supplementation significantly enhanced the mRNA expression of FGF-2, fibroblast growth factor rector 1 (FGFR-1), and FGFR-2 (FIG. 2A). No significant effect on the mRNA expression of FGFR-3 and FGFR-4 were found (FIG. 2A). Next, the mRNA expression of downstream targets of FGF signaling were assessed to determine the effect of FGF-8b supplementation on intracellular signaling. Several downstream targets of FGF signaling were found to be upregulated when ADSCs were supplemented with FGF-8b, demonstrating that exogenous FGF-8b supplementation effects signal transduction through the canonical pathway (FIG. 2B). Finally, FGF-8b supplementation (100 ng/mL) was found to upregulate the expression of an osteogenic (runx2) and chondrogenic marker of differentiation (sox9), while adipogenic (PPARg) and tenogenic (scx) markers were down regulated (FIG. 2C). Of note, myogenic marker (myoD) was only detected in FGF-8b supplementation (Table 2).

TABLE 2
qRT-PCR of myoD in rat ADSCs.

| | Ct value (gapdh) | Ct value (myoD) |
|---|---|---|
| Growth medium | 17.48 ± 0.29 | N/A |
| +FGF-8b (100 ng/mL) | 17.22 ± 0.30 | 34.76 ± 0.68 |

FGF-8 signaling demonstrates different MSC-fate decision compared to FGF-2. FGFs exert multifaceted effects through the activation of four distinct receptors (FGFR1-4) with differential binding properties, and the main downstream signaling pathways are the mitogen-activated protein kinase (RAS/MAPK) pathway, the phosphoinositide 3 (PI3) kinase/AKT pathway, and phospholipase C gamma (PLCγ) pathway (Yun et al., 2010). The result revealed FGF-8b promoted the expression of FGFR1 and 2 (FIG. 2A), which demonstrates that the FGF-8b supplementation can alter downstream FGF signaling. FGF-8b showed the opposite effect from FGF-2 in some lineages (adipogenesis, tenogenesis, and myogenesis) although FGF-2 expression was enhanced by FGF-8b supplementation (FIGS. 2A, 2C, 5, 6, and 7).

### Example 4: FGF-8b Enhances Chondrogenic Differentiation while Suppressing Tenogenic and Adipogenic Differentiation

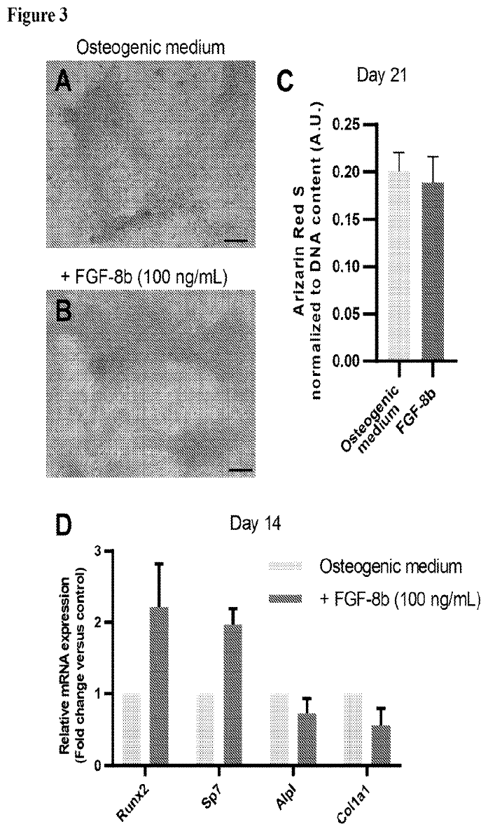

The effect of FGF-8b on the osteogenic, chondrogenic, adipogenic, and tenogenic differentiation of rADSCs was evaluated. First, osteogenic differentiation by was investigated by assessing the extent of calcium deposition and the expression of osteogenic markers of rADSCs with and without FGF-8b supplementation. FGF-8b supplementation did not significantly affect the calcium deposition of rADSCs both qualitatively and quantitatively (FIG. 3A-C). A trend towards the upregulation of osteogenic markers runx2 and sp7 was found (FIG. 3D). The results demonstrate that FGF-8b supplementation does not affect osteogenic differentiation in vitro.

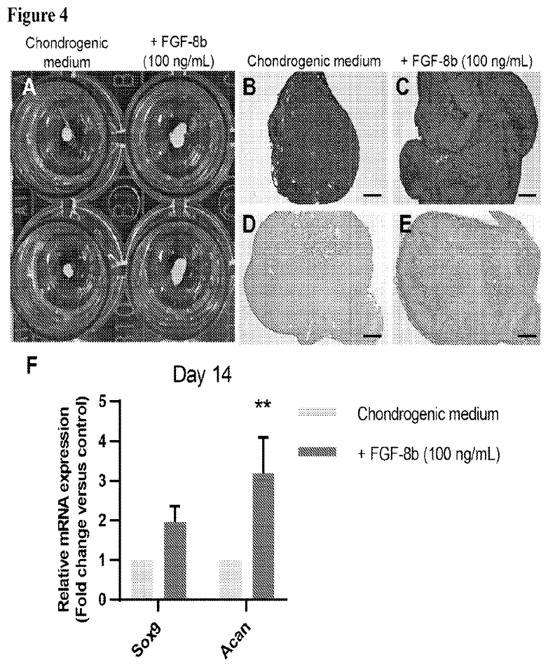

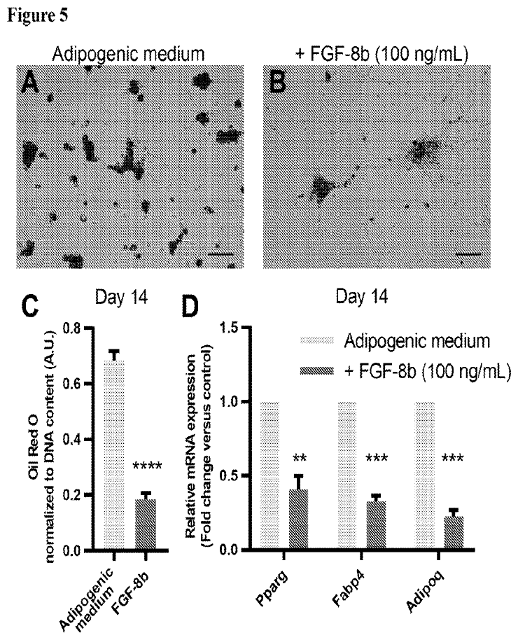

FGF-8b supplementation was found to enhance chondrogenic differentiation of micromass cultures of rat ADSCs. After 21 days of culture, the size of micromass cultures of rat ADSCs were qualitatively larger (FIG. 4A). FGF-8b increased the cellularity of micromass cultures as observed in sections stained with hematoxylin & cosin (FIGS. 4B & C). Additionally, the intensity of alcian staining was enhanced by FGF-8b supplementation, which is indicative of increased proteoglycan content within the extracellular matrix (FIG. 4 D &E). FGF-8b supplementation significantly enhanced the transcription of aggrecan at day 21, and a trend towards enhanced sox9 expression was observed (FIG. 4F). These results demonstrate that FGF-8b enhances micromass size through the proliferation of rat ADSCs and contributes to the development of a chondrogenic environment and transcriptome.

FGF-8b supplementation to adipogenic medium significantly inhibits rat ADSC adipogenesis when assessed at day 14. FGF-8b downregulated the formation of lipids as observed by oil O staining (FIGS. 5A & B). The accumulation and distribution of lipids in ADSCs was more robust in adipogenic medium alone (FIG. 5A), whereas the presence of lipids was sparse in the FGF-8b condition (FIG. 5B). This was confirmed by colorimetric analysis of extracted oil O from stained cultures (FIG. 5C). Transcriptionally, FGF-8b was found to significantly downregulate the adipogenic markers PPARg, fabp4, and adipoq (FIG. 5D). These results demonstrate that the exogenous administration of FGF-8b has a profound inhibitory effect on adipogenesis. FGF-8b supplementation to adipogenic medium suppress lipid production and the expression of adipogenic mRNA in rat ADSCs (FIG. 5).

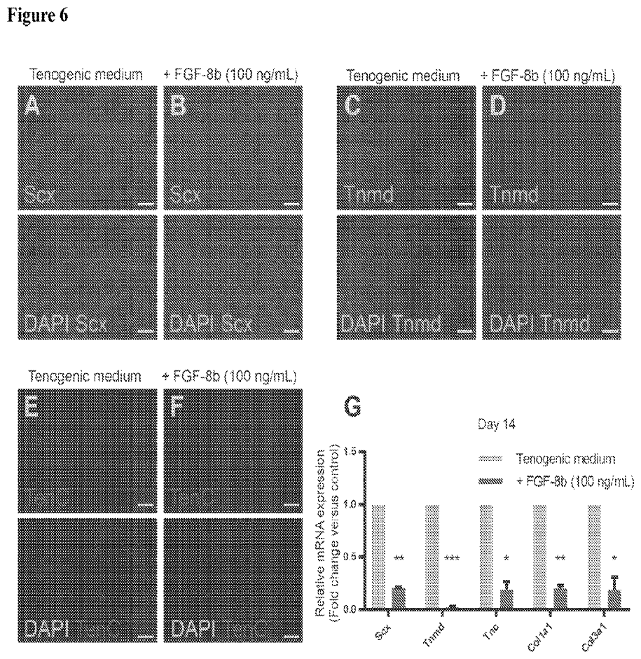

FGF-8b supplementation to tenogenic medium resulted in enhanced proliferation of ADSCs and inhibition of tenogenic differentiation. Immunostaining shows anisotropic organization of the confluent ADSC cultures (FIG. 6A-D). FGF-8b supplementation resulted in higher cellularity compared to tenogenic medium alone, as shown by nuclear staining (FIG. 6 A-D). The signal intensity of scleraxis and tenomodulin did not differ in either condition and was weak overall (FIG. 6A-D). Tenascin C staining was more robust in tenogenic medium alone (FIGS. 6E & F). FGF-8b was found to downregulate the expression of tenogenic markers (scx, tnmd, tenacin C) and tendon extracellular matrix proteins (collagen 1 and 2, FIG. 6G). These results desmonstrate that supplementation of FGF-8b in tenogenic medium leads to the proliferation of ADSCs rather than differentiation.

### Example 5: FGF-8b Enhances Myofiber Formation of Rat MPCs

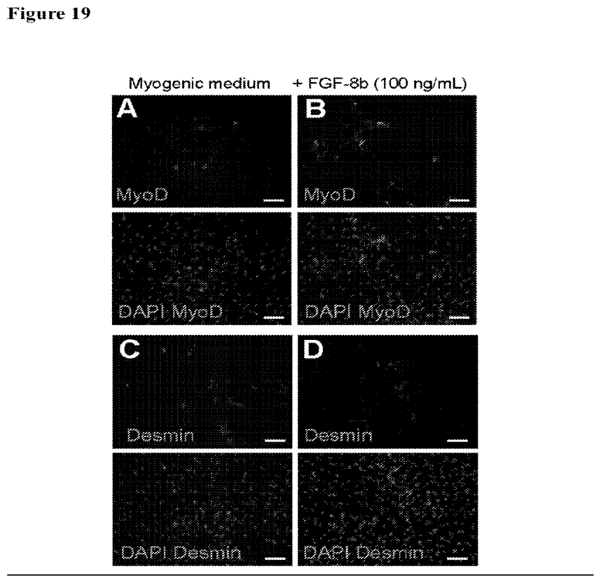

The low level of muscle specific proteins was assessed by immunofluorescence staining of MyoD and Desmin after 14 days of culture (FIG. 19). The effect of FGF-8b on myogenic differentiation was evaluated with rat MPCs.

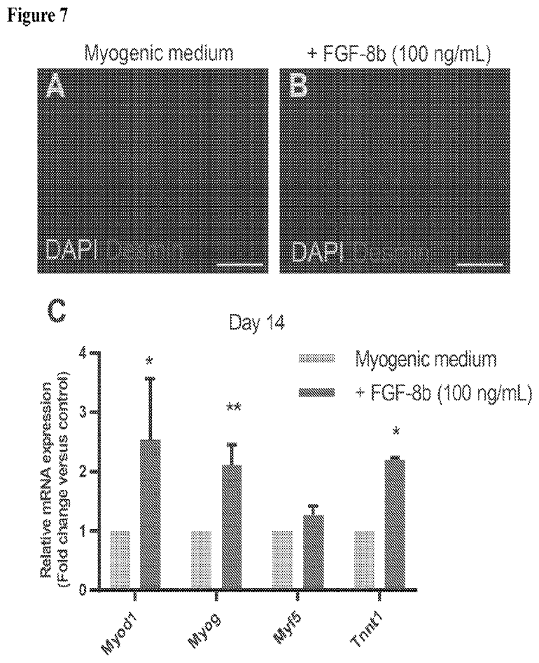

FGF-8b was found to enhance myofiber formation of MPCs as determined by immunostaining and qRT-PCR analysis. FGF-8b was found to enhance the presence of Desmin, the main intermediate filament of skeletal muscle (FIG. 7A, B). To determine the progression of myogenesis, qRT-PCR analysis was conducted. Data indicated that an early marker of myogenesis (myf5) was unchanged due to FGF-8b supplementation (FIG. 7C). However, mature myogenic markers (Myod1 & Myog) and slow skeletal muscle gene expression (Tnnt1) were upregulated in MPCs (FIG. 7C). These data demonstrate that FGF-8b supplementation to myogenic medium significantly contribute to MPC maturation.

FGF-8 signaling in somite myogenesis is recapitulated in vitro with muscle progenitor cells. FGF-8 had opposing effects on tendon and muscle gene expression (FIG. 6F and FIG. 7C). Tendon markers of ADSCs were found to be downregulated in the presence of FGF-8 supplementation demonstrating that FGF-8 is involved in the maintenance of mature tendon rather than the induction of tenogenesis. Likewise, FGF-8b treatment of ADSCs in myogenic induction medium did not affect the presence of muscle fiber proteins (FIG. 19). FGF-8b was found to enhance the expression of myoD and myogenin.

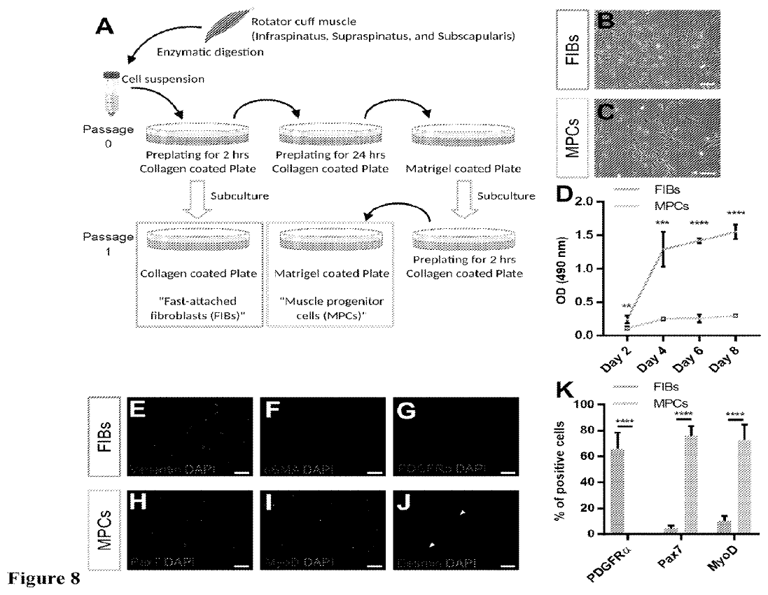

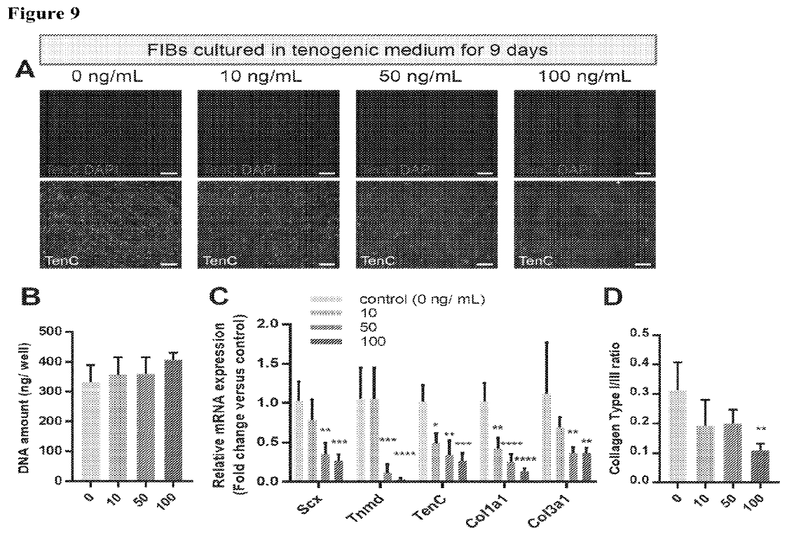

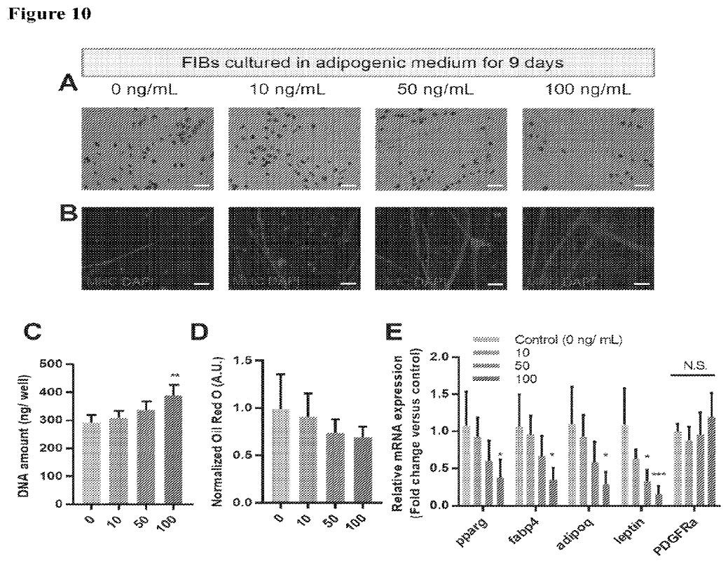

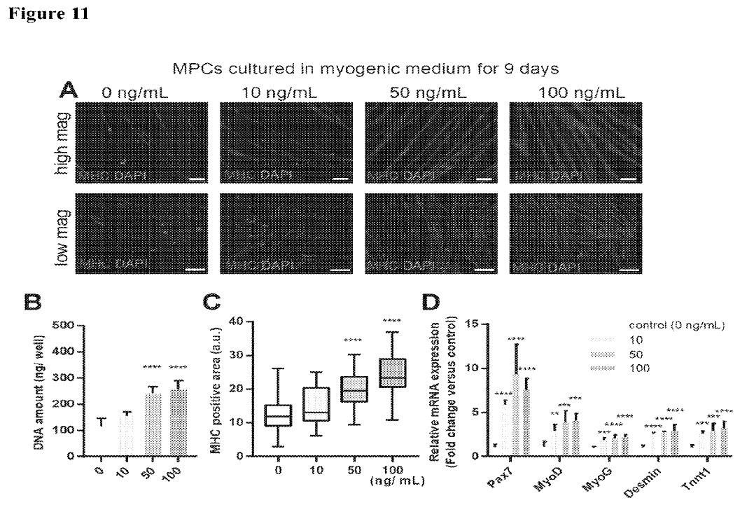

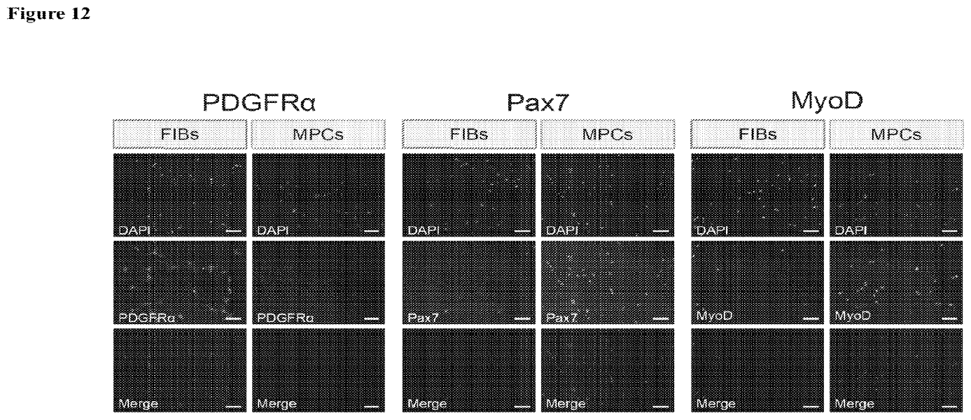

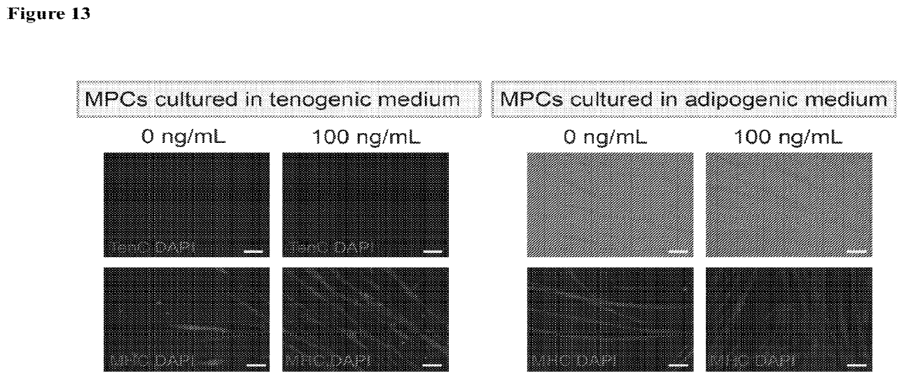

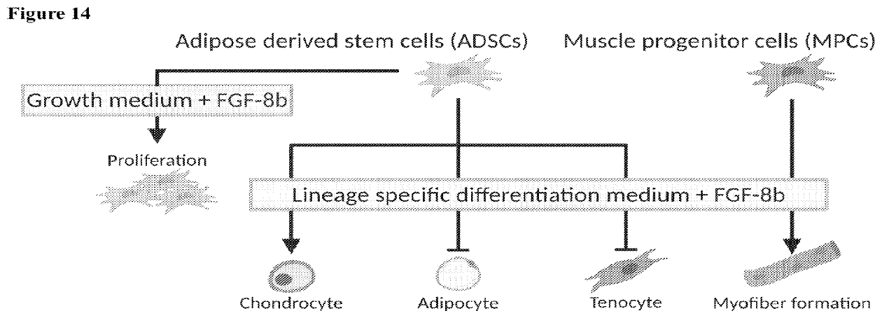

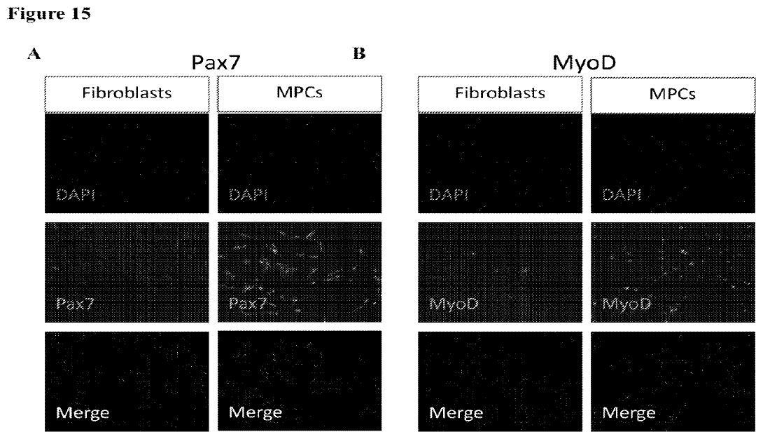

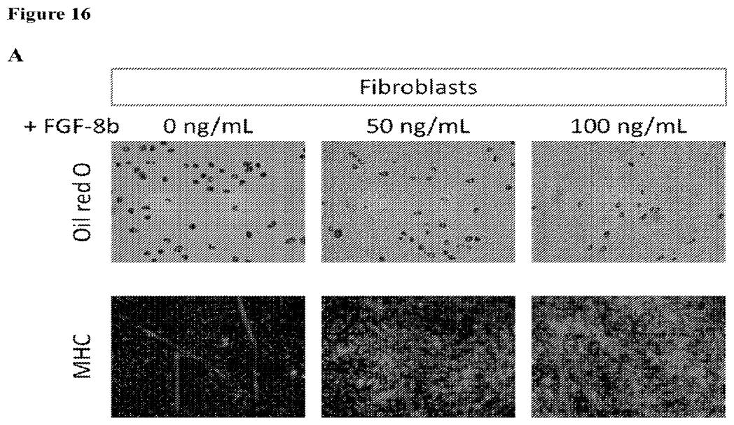

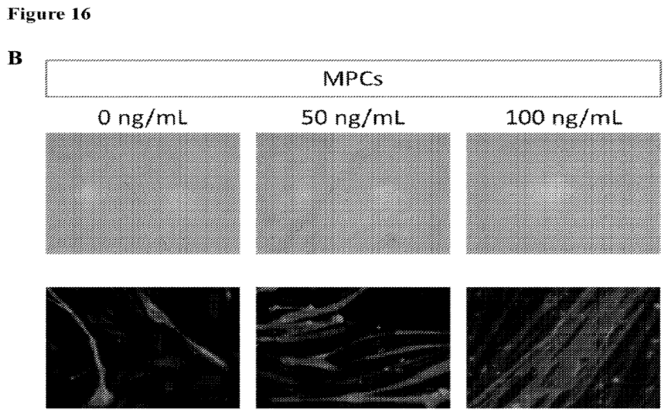

The rotator cuff muscle cells were separated into two populations: fibro-adipogenic progenitors (FAPs)-rich fibroblasts (FIBs) and satellite stem cells (SCs)-rich muscle progenitor cells (MPCs) (FIG. 8). FIBs and MPCs shows different cell morphology and different metabolic activities (FIG. 8B-D). FIBs are positive for fibroblast markers (Vimentin and αSMA) and FAP marker (FIG. 8E-G). MPCs are positive for satellite stem cell marker (Pax7), myo-progenitor cell marker (MyoD), but not positive for Myocyte marker (Desmin) that shows MPCs retain undifferentiated status. FIG. 8K and FIG. 12 shows the enrichment of FAPs in FIBs and SCs in MPCs. FIG. 9A shows the results of suppressed tenogenic differentiation in FIBs by FGF-8b treatment. FGF-8b treatment shows the trend of cell proliferation, but not statistically significant (FIG. 9B) The result of qRT-PCR shows tenogenic marker genes expression is suppressed according to the concentration of FGF-8b. The ratio of Collagen type I and III expression suggests FGF8b showed an anti-fibrotic response. Adipogenic differentiation was suppressed, but myofiber formation is enhanced in FIBs (FIG. 10A-D). Adipogenic marker gene expression is suppressed according to the FGF-8b concentration, but FAP marker gene (PDGFRα) expression is not affected (FIG. 10E). Myogenic differentiation in MPCs was enhanced (FIG. 11). Not only myogenic marker genes but also satellite stem cell marker gene (Pax7) are upregulated according to the FGF-8b concentration (FIG. 11D). Representative images of FIBs and MPCs staining related to FIG. 8, are shown in FIG. 12. MPCs did not differentiate into tenocytes and adipocytes (FIG. 13).

The results demonstrate that micromass cultures of ADSCs are enhanced in size and have increased proteoglycan content. Furthermore, on the molecular level chondrocyte markers were enhanced when ADSC chondrogenic differentiation medium was supplemented with FGF-8b. This demonstrates that inguinal ADSCs chondrogenic potential is enhanced by FGF-8b.

Exogenous supplementation of FGF-8b has fundamental effects on the differentiation potential of ADSCs and MPCs. The data shows that FGF-8b supplementation enhances ADSCs and MPCs proliferation in all differentiation medium conditions assessed. The data also demonstrates that FGF-8b supplementation can also upregulate or downregulate differentiation markers of ADSCs and MPCs. FGF-8b supplementation 1) activates the FGF signaling pathway, 2) inhibits adipogenic and tenogenic differentiation, and 3) enhances chondrogenic and myogenic differentiation. FGF-8b did not affect osteogenic differentiation.

The findings on the fundamental roles of exogenous FGF-8b on MSCs, demonstrates its diverse biological function for tissue regeneration and provides a basis to engineer therapies for musculoskeletal applications. Based on the data, there are two potential orthopedic applications for FGF-8b: 1) Downregulation of adipose induced inflammation in the knee joint for the treatment of osteoarthritis, and 2) regeneration of rotator cuff tissue.

The present findings that FGF-8b inhibits adipose differentiation and promotes chondrogenesis makes it a candidate to treat osteoarthritis. FGF-8b is also a candidate to administer during the repair of rotator cuff tears due to its pro-myogenic effect on MPCs and anti-adipogenesis effect on MSCs.

### REFERENCES

Abramoff, B., Caldera, F. E., 2020. Osteoarthritis: Pathology, Diagnosis, and Treatment Options. Med. Clin. North Am., Physical Medicine and Rehabilitation: An Update for Internists. https://doi.org/10.1016/j.mcna.2019.10.007
Ahn, H. J., Lee, W. J., Kwack, K. B., Kwon, Y. Do, 2009. FGF2 stimulates the proliferation of human mesenchymal stem cells through the transient activation of JNK signaling. FEBS Lett. 583, 2922-2926. https://doi.org/10.1016/j.febslet.2009.07.056
Bobick, B. E., Thornhill, T. M., Kulyk, W. M., 2007. Fibroblast growth factors 2, 4, and 8 exert both negative and positive effect on limb, frontonasal, and mandibular chondrogenesis via MEK-ERK activation. J. Cell. Physiol. 211, 233-243. https://doi.org/10.1002/jcp.20923
Brockes, J. P., Kumar, A., 2005. Appendage regeneration in adult vertebrates and implications for regenerative medicine. Science 310, 1919-23. https://doi.org/10.1126/science.1115200
Cai, T. Y., Zhu, W., Chen, X. S., Zhou, S. Y., Jia, L. S., Sun, Y. Q., 2013. Fibroblast growth factor 2 induces mesenchymal stem cells to differentiate into tenocytes through the MAPK pathway. Mol. Med. Rep. 8, 1323-1328. https://doi.org/10.3892/mmr.2013.1668
Chamberlain, G., Fox, J., Ashton, B., Middleton, J., 2007. Concise Review: Mesenchymal Stem Cells: Their Phenotype, Differentiation Capacity, Immunological Features, and Potential for Homing. Stem Cells 25, 2739-2749. https://doi.org/10.1634/stemcells.2007-0197
Chiou, M., Xu, Y., Longaker, M. T., 2006. Mitogenic and chondrogenic effects of fibroblast growth factor-2 in adipose-derived mesenchymal cells. Biochem. Biophys. Res. Commun. 343, 644-652. https://doi.org/10.1016/j.bbrc.2006.02.171
Christensen, R. N., Weinstein, M., Tassava, R. A., 2002. Expression of fibroblast growth factors 4, 8, and 10 in limbs, flanks, and blastemas of Ambystoma. Dev. Dyn. 223, 193-203. https://doi.org/10.1002/dvdy. 10049
Correa, D., Somoza, R. A., Lin, P., Greenberg, S., Rom, E., Duesler, L., Welter, J. F., Yayon, A., Caplan, A. I., 2015. Sequential exposure to fibroblast growth factors (FGF) 2, 9 and 18 enhances hMSC chondrogenic differentiation. Osteoarthr. Cartil. 23, 443-453. https://doi.org/10.1016/j.joca.2014.11.013
Crossley, P. H., Minowada, G., MacArthur, C. A., Martin, G. R., 1996. Roles for FGF8 in the induction, initiation, and maintenance of chick limb development. Cell 84, 127-136. https://doi.org/10.1016/S0092-8674(00)80999-X
Di Rocco, G., Iachininoto, M. G., Tritarelli, A., Straino, S., Zacheo, A., Germani, A., Crea, F., Capogrossi, M. C., 2006. Myogenic potential of adipose-tissue-derived cells. J. Cell Sci. 119, 2945-2952. https://doi.org/10.1242/jcs.03029
Edom-Vovard, F., Bonnin, M. A., Duprez, D., 2001. Fgf8 transcripts are located in tendons during embryonic chick limb development. Mech. Dev. 108, 203-206. https://doi.org/10.1016/S0925-4773(01)00483-X
Farkas, J. E., Monaghan, J. R., 2017. A brief history of the study of nerve dependent regeneration. Neurogenesis. https://doi.org/10.1080/23262133.2017.1302216
Gladstone, J. N., Bishop, J. Y., Lo, I. K. Y., Flatow, E. L., 2007. Fatty infiltration and atrophy of the rotator cuff do not improve after rotator cuff repair and correlate with poor functional outcome. Am. J. Sports Med. 35, 719-728. https://doi.org/10.1177/0363546506297539
Greif, D. N., Kouroupis, D., Murdock, C. J., Griswold, A. J., Kaplan, L. D., Best, T. M., Correa, D., 2020. Infrapatellar Fat Pad/Synovium Complex in Early-Stage Knee Osteoarthritis: Potential New Target and Source of Therapeutic Mesenchymal Stem/Stromal Cells. Front. Bioeng. Biotechnol. 8. https://doi.org/10.3389/fbioe.2020.00860
Groves, J. A., Hammond, C. L., Hughes, S. M., 2005. Fgf8 drives myogenic progression of a novel lateral fast muscle fibre population in zebrafish. Development 132, 4211-4222. https://doi.org/10.1242/dev.01958
Han, M.-J., An, J.-Y., Kim, W.-S., 2001. Expression patterns of Fgf-8 during development and limb regeneration of the axolotl. Dev. Dyn. 220, 40-48. https://doi.org/10.1002/1097-0177(2000)9999:99993.0.CO:2-8
Han, M., Yang, X., Taylor, G., Burdsal, C. A., Anderson, R. A., Muneoka, K., 2005. Limb regeneration in higher vertebrates: Developing a roadmap. Anat. Rec.-Part B New Anat. 287, 14-24. https://doi.org/10.1002/ar.b.20082
Hanada, K., Dennis, J. E., Caplan, A. I., 1997. Stimulatory effects of basic fibroblast growth factor and bone morphogenetic protein-2 on osteogenic differentiation of rat bone marrow-derived mesenchymal stem cells. J. Bone Miner. Res. 12, 1606-1614. https://doi.org/10.1359/jbmr. 1997.12.10.1606
Huang, Z., Ren, P. G., Ma, T., Smith, R. L., Goodman, S. B., 2010. Modulating osteogenesis of mesenchymal stem cells by modifying growth factor availability. Cytokine 51, 305-310. https://doi.org/10.1016/j.cyto.2010.06.002
Hutley, L. J., Newell, F. S., Kim, Y. H., Luo, X., Widberg, C. H., Shurety, W., Prins, J. B., Whitehead, J. P., 2011. A putative role for endogenous FGF-2 in FGF-1 mediated differentiation of human preadipocytes. Mol. Cell. Endocrinol. 339, 165-171. https://doi.org/10.1016/j.mce.2011.04.012
James, R., Mengsteab, P., Laurencin, C. T., 2016. Regenerative Engineering: Studies of the Rotator Cuff and other Musculoskeletal Soft Tissues, in: MRS Advances. pp. 1255-1263. https://doi.org/10.1557/adv.2016.282
Kakudo, N., Shimotsuma, A., Kusumoto, K., 2007. Fibroblast growth factor-2 stimulates adipogenic differentiation of human adipose-derived stem cells. Biochem. Biophys. Res. Commun. 359, 239-244. https://doi.org/10.1016/j.bbrc.2007.05.070
Kawakami, Y., Rodríguez-León, J., Koth, C. M., Büscher, D., Itoh, T., Raya, Á., Ng, J. K., Esteban, C. R., Takahashi, S., Henrique, D., Schwarz, M. F., Asahara, H., Izpisúa Belmonte, J. C., 2003. MKP3 mediates the cellular response to FGF8 signalling in the vertebrate limb. Nat. Cell Biol. https://doi.org/10.1038/ncb989
Kim, S., Ahn, C., Bong, N., Choe, S., Lee, D. K., 2015. Biphasic effects of FGF2 on adipogenesis. PLOS One 10, e0120073. https://doi.org/10.1371/journal.pone.0120073
Kragl, M., Knapp, D., Nacu, E., Khattak, S., Maden, M., Epperlein, H. H., Tanaka, E. M., 2009. Cells keep a memory of their tissue origin during axolotl limb regeneration. Nature 460, 60-65. https://doi.org/10.1038/nature08152
Lai, W. T., Krishnappa, V., Phinney, D. G., 2011. Fibroblast growth factor 2 (Fgf2) inhibits differentiation of mesenchymal stem cells by inducing Twist2 and Spry4, blocking extracellular regulated kinase activation, and altering fgf receptor expression levels. Stem Cells 29, 1102-1111. https://doi.org/10.1002/stem.661
Lansdown, D. A., Lee, S., Sam, C., Krug, R., Feeley, B. T., Ma, C. B., 2017. A prospective, Quantitative evaluation of fatty infiltration before and after rotator cuff repair. Orthop. J. Sport. Med. 5. https://doi.org/10.1177/2325967117718537
Laurencin, C. T., Khan, Y., 2012. Regenerative Engineering. Sci. Transl. Med. 4, 160ed9-160ed9. https://doi.org/10.1126/scitranslmed.3004467
Laurencin, C. T., Nair, L. S., 2016. The Quest toward limb regeneration: a regenerative engineering approach. Regen. Biomater. 3, 123-125. https://doi.org/10.1093/rb/rbw002
Laurencin, C. T., Nair, L. S., 2015. Regenerative Engineering: Approaches To Limb Regeneration and Other Grand Challenges. Regen. Eng. Transl. Med. 1, 1-3. https://doi.org/10.1007/s40883-015-0006-z
Lewandoski, M., Sun, X., Martin, G. R., 2000. Fgf8 signalling from the AER is essential for normal limb development. Nat. Genet. 26, 460-463. https://doi.org/10.1038/82609
Li, C., Scott, D. A., Hatch, E., Tian, X., Mansour, S. L., 2007. Dusp6 (Mkp3) is a negative feedback regulator of FgF-stimulated ERK signaling during mouse development. Development 134, 167-176. https://doi.org/10.1242/dev.02701
Maddaluno, L., Urwyler, C., Werner, S., 2017. Fibroblast growth factors: Key players in regeneration and tissue repair. Dev. https://doi.org/10.1242/dev.152587
Makanae, A., Mitogawa, K., Satoh, A., 2014. Co-operative Bmp- and Fgf-signaling inputs convert skin wound healing to limb formation in urodele amphibians. Dev. Biol. 396, 57-66. https://doi.org/10.1016/j.ydbio.2014.09.021
Mao, J., McGlinn, E., Huang, P., Tabin, C. J., McMahon, A. P., 2009. Fgf-Dependent Etv4/5 Activity Is Required for Posterior Restriction of Sonic hedgehog and Promoting Outgrowth of the Vertebrate Limb. Dev. Cell 16, 600-606. https://doi.org/10.1016/j.devcel.2009.02.005
Martin, G. R., 1998. The roles of FGFs in the early development of vertebrate limbs. Genes Dev. https://doi.org/10.1101/gad. 12.11.1571
Masaki, H., Ide, H., 2007. Regeneration potency of mouse limbs. Dev. Growth Differ. 49, 89-98. https://doi.org/10.1111/j. 1440-169X.2007.00909.x
McCusker, C., Bryant, S. V., Gardiner, D. M., 2015. The axolotl limb blastema: cellular and molecular mechanisms driving blastema formation and limb regeneration in tetrapods. Regeneration 2, 54-71. https://doi.org/10.1002/reg2.32
McCusker, C. D., Diaz-Castillo, C., Sosnik, J., Phan, A., Gardiner, D. M., 2016. Cartilage and bone cells do not participate in skeletal regeneration in Ambystoma mexicanum limbs. Dev. Biol. https://doi.org/10.1016/j.ydbio.2016.05.032
Mengsteab, P. Y., Otsuka, T., McClinton, A., Shemshaki, N. S., Shah, S., Kan, H.-M., Obopilwe, E., Vella, A. T., Nair, L. S., Laurencin, C. T., 2020. Mechanically superior matrices promote osteointegration and regeneration of anterior cruciate ligament tissue in rabbits. Proc. Natl. Acad. Sci. 117, 28655-28666. https://doi.org/10.1073/pnas.2012347117
Minetto, M. A., Giannini, A., McConnell, R., Busso, C., Torre, G., Massazza, G., 2020. Common Musculoskeletal Disorders in the Elderly: The Star Triad. J. Clin. Med. 9, 1216. https://doi.org/10.3390/jcm9041216
Minowada, G., Jarvis, L. A., Chi, C. L., Neubuser, A., Sun, X., Hacohen, N., Krasnow, M. A., Martin, G. R., 1999. Vertebrate Sprouty genes are induced by FGF signaling and can cause chondrodysplasia when overexpressed. Development 126, 4465-4475.
Nacu, E., Gromberg, E., Oliveira, C. R., Drechsel, D., Tanaka, E. M., 2016. FGF8 and SHH substitute for anterior-posterior tissue interactions to induce limb regeneration. Nature 1, 1-16. https://doi.org/10.1038/nature17972
Neubauer, M., Fischbach, C., Bauer-Kreisel, P., Lieb, E., Hacker, M., Tessmar, J., Schulz, M. B., Goepferich, A., Blunk, T., 2004. Basic fibroblast growth factor enhances PPARy ligand-induced adipogenesis of mesenchymal stem cells. FEBS Lett. 577, 277-283. https://doi.org/10.1016/j.febslet.2004.10.020
Norelli, J. B., Plaza, D. P., Stal, D. N., Varghese, A. M., Liang, H., Grande, D. A., 2018. Tenogenically differentiated adipose-derived stem cells are effective in Achilles tendon repair in vivo. J. Tissue Eng. 9. https://doi.org/10.1177/2041731418811183
Olwin, B. B., Rapraeger, A., 1992. Repression of myogenic differentiation by aFGF, bFGF, and K-FGF is dependent on cellular heparan sulfate. J. Cell Biol. 118, 631-639. https://doi.org/10.1083/jcb.118.3.631
Ornitz, D. M., Marie, P. J., 2015. Fibroblast growth factor signaling in skeletal development and disease. Genes Dev. https://doi.org/10.1101/gad.266551.115
Osti, L., Buda, M., Del Buono, A., 2013. Fatty infiltration of the shoulder: Diagnosis and reversibility. Muscles. Ligaments Tendons J. https://doi.org/10.11138/mltj/2013.3.4.351
Pittenger, M. F., Mackay, A. M., Beck, S. C., Jaiswal, R. K., Douglas, R., Mosca, J. D., Moorman, M. A., Simonetti, D. W., Craig, S., Marshak, D. R., 1999. Multilineage potential of adult human mesenchymal stem cells. Science (80-.). 284, 143-147. https://doi.org/10.1126/science.284.5411.143
Purushothaman, S., Elewa, A., Seifert, A. W., 2019. Fgf-signaling is compartmentalized within the mesenchyme and controls proliferation during salamander limb development. Elife 8, e48507. https://doi.org/10.7554/eLife.48507
Satoh, A., makanae, A., Hirata, A., Satou, Y., 2011. Blastema induction in aneurogenic state and Prrx-1 regulation by MMPs and FGFs in Ambystoma mexicanum limb regeneration. Dev. Biol. 355, 263-274. https://doi.org/10.1016/j.ydbio.2011.04.017
Satoh, A., Makanae, A., Nishimoto, Y., Mitogawa, K., 2016. FGF and BMP derived from dorsal root ganglia regulate blastema induction in limb regeneration in Ambystoma mexicanum. Dev. Biol. 417, 114-125. https://doi.org/10.1016/j.ydbio.2016.07.005
Satoh, A., Mitogawa, K., Makanae, A., 2018. Nerve roles in blastema induction and pattern formation in limb regeneration. Int. J. Dev. Biol. https://doi.org/10.1387/ijdb.180118as
Schmidt, L., Taiyab, A., Melvin, V. S., Jones, K. L., Williams, T., 2018. Increased FGF8 signaling promotes chondrogenic rather than osteogenic development in the embryonic skull. DMM Dis. Model. Mech. 11. https://doi.org/10.1242/dmm.031526
Shamsi, F., Xue, R., Huang, T. L., Lundh, M., Liu, Y., Leiria, L. O., Lynes, M. D., Kempf, E., Wang, C. H., Sugimoto, S., Nigro, P., Landgraf, K., Schulz, T., Li, Y., Emanuelli, B., Kothakota, S., Williams, L. T., Jessen, N., Pedersen, S. B., Böttcher, Y., Blüher, M., Körner, A., Goodyear, L. J., Mohammadi, M., Kahn, C. R., Tseng, Y. H., 2020. FGF6 and FGF9 regulate UCP1 expression independent of brown adipogenesis. Nat. Commun. 11, 1421. https://doi.org/10.1038/s41467-020-15055-9
Singer, M., 1978. On the Nature of the Neurotrophic Phenomenon in Urodele Limb Regeneration. Integr. Comp. Biol. 18, 829-841. https://doi.org/10.1093/icb/18.4.829
Solchaga, L. A., Penick, K., Porter, J. D., Goldberg, V. M., Caplan, A. I., Welter, J. F., 2005. FGF-2 enhances the mitotic and chondrogenic potentials of human adult bone marrow-derived mesenchy mal stem cells. J. Cell. Physiol. 203, 398-409. https://doi.org/10.1002/jcp.20238
Stocum, D. L., 2017. Mechanisms of urodele limb regeneration. Regeneration 159-200. https://doi.org/10.1002/reg2.92
Sun, X., Lewandoski, M., Meyers, E. N., Liu, Y. H., Maxson, R. E., Martin, G. R., 2000. Conditional inactivation of Fgf4 reveals complexity of signalling during limb bud development. Nat. Genet. 25, 83-86. https://doi.org/10.1038/75644
Taghiyar, L., Hesaraki, M., Sayahpour, F. A., Satarian, L., Hosseini, S., Aghdami, N., Eslaminejad, M. B., 2017. Msh homeobox 1 (Msx1)- and Msx2-overexpressing bone marrow-derived mesenchymal stem cells resemble blastema cells and enhance regeneration in mice. J. Biol. Chem. 292, 10520-10533. https://doi.org/10.1074/jbc.M116.774265
Tanaka, E. M., 2016. The Molecular and Cellular Choreography of Appendage Regeneration. Cell. https://doi.org/10.1016/j.cell.2016.05.038
Teven, C. M., Farina, E. M., Rivas, J., Reid, R. R., 2014. Fibroblast growth factor (FGF) signaling in development and skeletal diseases. Genes Dis. https://doi.org/10.1016/j.gendis.2014.09.005
Uchii, M., Tamura, T., Suda, T., Kakuni, M., Tanaka, A., Miki, I., 2008. Role of fibroblast growth factor 8 (FGF8) in animal models of osteoarthritis. Arthritis Res. Ther. 10, R90. https://doi.org/10.1186/ar2474
Wang, R., Liu, W., Du, M., Yang, C., Li, X., Yang, P., 2018. The differential effect of basic fibroblast growth factor and Stromal cell-derived factor-1 pretreatment on bone morrow mesenchymal stem cells osteogenic differentiation potency. Mol. Med. Rep. 17, 3715-3721. https://doi.org/10.3892/mmr.2017.8316
Westphal, S., Gantert, T., Kless, C., Hüttinger, K., Klingenspor, M., Fromme, T., 2019. Fibroblast growth factor 8b induces uncoupling protein 1 expression in epididymal white preadipocytes. Sci. Rep. 9, 8470. https://doi.org/10.1038/s41598-019-44878-w
Widberg, C. H., Newell, F. S., Bachmann, A. W., Ramnoruth, S. N., Spelta, M. C., Whitehead, J. P., Hutley, L. J., Prins, J. B., 2009. Fibroblast growth factor receptor 1 is a key regulator of early adipogenic events in human preadipocytes. Am. J. Physiol.-Endocrinol. Metab. 296, E121-E131. https://doi.org/10.1152/ajpendo.90602.2008
Xu, X., Weinstein, M., Li, C., Naski, M., Cohen, R. I., Ornitz, D. M., Leder, P., Deng, C., 1998. Fibroblast growth factor receptor 2 (FGFR2)-mediated reciprocal regulation loop between FGF8 and FGF 10 is essential for limb induction. Development 125, 753-765.
Yablonka-Reuveni, Z., Danoviz, M. E., Phelps, M., Stuelsatz, P., 2015. Myogenic-specific ablation of Fgfrl impairs FGF2-mediated proliferation of satellite cells at the myofiber niche but does not abolish the capacity for muscle regeneration. Front. Aging Neurosci. 7. https://doi.org/10.3389/fnagi.2015.00085
Yokoyama, H., 2008. Initiation of limb regeneration: The critical steps for regenerative capacity. Dev. Growth Differ. https://doi.org/10.1111/j.1440-169X.2007.00973.x
Yokoyama, H., Ide, H., Tamura, K., 2001. FGF-10 stimulates limb regeneration ability in Xenopus laevis. Dev. Biol. 233, 72-79. https://doi.org/10.1006/dbio.2001.0180
Yu, K., Ornitz, D. M., 2008. FGF signaling regulates mesenchymal differentiation and skeletal patterning along the limb bud proximodistal axis. Development 135, 483-491. https://doi.org/10.1242/dev.013268
Yu, X., Mengsteab, P. Y., Narayanan, G., Nair, L. S., Laurencin, C. T., 2020. Enhancing the Surface Properties of a Bioengineered Anterior Cruciate Ligament Matrix for Use with Point-of-Care Stem Cell Therapy. Engineering. https://doi.org/10.1016/j.eng.2020.02.010
Yun, Y. R., Won, J. E., Jeon, E., Lee, S., Kang, W., Jo, H., Jang, J. H., Shin, U. S., Kim, H. W., 2010. Fibroblast growth factors: Biology, function, and application for tissue regeneration. J. Tissue Eng. https://doi.org/10.4061/2010/218142
Zaragosi, L.-E., Ailhaud, G., Dani, C., 2006. Autocrine Fibroblast Growth Factor 2 Signaling Is Critical for Self-Renewal of Human Multipotent Adipose-Derived Stem Cells. Stem Cells 24, 2412-2419. https://doi.org/10.1634/stemcells.2006-0006
Zhang, Z., Sui, P., Dong, A., Hassell, J., Cserjesi, P., Chen, Y. T., Behringer, R. R., Sun, X., 2010. Preaxial polydactyly: Interactions among ETV, TWIST1 and HAND2 control anterior-posterior patterning of the limb. Development 137, 3417-3426. https://doi.org/10.1242/dev.051789
Zhou, J., Meng, J., Guo, S., Gao, B., Ma, G., Zhu, X., Hu, J., Xiao, Y., Lin, C., Wang, H., Ding, L., Feng, G., Guo, X., He, L., 2007. IHH and FGF8 coregulate elongation of digit primordia. Biochem. Biophys. Res. Commun. 363, 513-518. https://doi.org/10.1016/j.bbrc.2007.08.198

1. A method for enhancing myogenic and/or chondrogenic lineage commitment of mammalian mesenchymal stem cells (MSCs), including but not limited to adipose derived stem cells (ADSCs), and/or muscle progenitor cells (MPCs), comprising contacting the MSCs, ADSCs, and/or MPCs with an amount effective of fibroblast growth factor 8 (FGF8) to enhance myogenic and/or chondrogenic lineage commitment of the MSCs, ADSCs, and/or MPCs.

2. The method according to claim 1, wherein the method comprises contacting MSCs, ADSCs, and/or MPCs with an amount effective of fibroblast growth factor 8 (FGF8) to enhance myogenic and/or chondrogenic lineage commitment of the MSCs, ADSCs, and/or MPCs.

3-4. (canceled)

5. A method for suppressing adipogenic and/or tenogenic differentiation of MSCs, including but not limited to ADSCs, and/or MPCs, comprising contacting mammalian MSCs, ADSCs, and/or MPCs with an amount effective of fibroblast growth factor 8 (FGF8) to suppress adipogenic and/or tenogenic differentiation of the MSCs, ADSCs, and/or MPCs.

6. The method according to claim 5, wherein the method comprises contacting mammalian MSCs, ADSCs, and/or MPCs with an amount effective of fibroblast growth factor 8 (FGF8) to suppress adipogenic and/or tenogenic differentiation of the MSCs, ADSCs, and/or MPCs.

7-8. (canceled)

9. The method of claim 1, wherein the contacting occurs in vitro.

10. The method of claim 9, wherein the in vitro contacting comprises growth medium, osteogenic medium, adipogenic medium, and/or chondrogenic medium.

11. The methods of claim 9, wherein the mammalian MSCs, ADSCs, and/or MPCs are human, mouse, rabbit, or horse MSCs, ADSCs, and/or MPCs

12. The method of claim 1, wherein the contacting occurs in vivo in a mammalian subject.

13. The method of claim 12, wherein the method promotes tissue regeneration, myofiber formation, and/or myogenesis in the mammalian subject.

14. The method of claim 12, wherein the mammalian subject has a musculoskeletal disorder, osteoarthritis, a rotator cuff injury, and/or a paraspinal injury.

15. (canceled)

16. The method of claim 14, wherein, the mammalian subject has osteoarthritis, and the method results in downregulation of adipose induced inflammation in the knee joint, to treat the osteoarthritis.

17. The method of claim 16, wherein the contacting comprises administering the FGF8 to or proximal to the knee joint, or to fat pads of the knee joint.

18. (canceled)

19. The method of claim 14, wherein, the mammalian subject has a rotator cuff injury, and the contacting comprises administering the FGF8 to or proximal to the rotator cuff.

20. (canceled)

21. The method of claim 14, wherein, the mammalian subject has a paraspinal injury, and the method results in downregulation of adipose induced inflammation in the spine.

22. The method of claim 21, wherein the contacting comprises administering the FGF8 to the spine or to fat pads of the spine.

23. The method of claim 1, where the FGF8 comprises human or mouse FGF8b.

24-26. (canceled)

27. A method for treating a musculoskeletal disorder, comprising administering an amount effective of fibroblast growth factor 8 (FGF8) to a mammalian subject having a musculoskeletal disorder, to treat the disorder.

28. The method of claim 27, wherein the mammalian subject is a human subject.

29-36. (canceled)

37. The method of claim 1, wherein the FGF8 comprises a polypeptide according to any one of SEQ ID NOs: 1-7, or a fragment thereof.

38-39. (canceled)

40. The method of claim 1, wherein the FGF8 comprises
(a) residues 23-209 of SEQ ID NOs: 1 or 4, optionally including an N-terminal methionine;
(b) residues 23-204 of SEO ID NOs: 2, optionally including an N-terminal methionine;
(c) residues 23-215 of SEO ID NOs: 3, 5, or 7, optionally including an N-terminal methionine; and/or
(d) residues 23-190 of SEO ID NOs: 6, optionally including an N-terminal methionine.

41-42. (canceled)


SEQUENCE LISTING (7 sequences)

SEQ ID NO: 1 - protein, 233 aa, Homo sapiens
  MISC_FEATURE (1)..(1): optionally absent
MGSPRSALSCLLLHLLVLCLQAQEGPGRGPALGRELASLFRAGREPQGVSQQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR

SEQ ID NO: 2 - protein, 204 aa, Homo sapiens
  MISC_FEATURE (1)..(1): optionally absent
MGSPRSALSCLLLHLLVLCLQAQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR

SEQ ID NO: 3 - protein, 215 aa, Homo sapiens
  MISC_FEATURE (1)..(1): optionally absent
MGSPRSALSCLLLHLLVLCLQAQVTVQSSPNFTQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR

SEQ ID NO: 4 - protein, 244 aa, Homo sapiens
  MISC_FEATURE (1)..(1): optionally absent
MGSPRSALSCLLLHLLVLCLQAQEGPGRGPALGRELASLFRAGREPQGVSQQVTVQSSPNFTQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR

SEQ ID NO: 5 - protein, 215 aa, Mus musculus; identical to SEQ ID NO: 3
  MISC_FEATURE (1)..(1): optionally absent

SEQ ID NO: 6 - protein, 197 aa, Oryctolagus cuniculus
  MISC_FEATURE (1)..(1): optionally absent
MGSPRSALSCLLLHLLVLCLQAQVTVQSSPNFTQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYP

SEQ ID NO: 7 - protein, 270 aa, Equus caballus
  MISC_FEATURE (1)..(1): optionally absent
MGSPRSALSCLLLHLLVLCLQAQVRRTARRRGPGGRRGTPVDNVGQGHEDGPCGRRGRAGRNLSSPAPNYPEEGAKEQRGSALPKVTQQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR